(19) United States
(12) Patent Application Publication
(10) Pub. No.: US 2019/0143021 A1
(43) Pub. Date: May 16, 2019

(54) ORGAN RESTRAINT FOR INFLAMMATION REDUCTION AND ATRIAL FIBRILLATION PREVENTION

(71) Applicant: Edwards Lifesciences Corporation, Irvine, CA (US)

(72) Inventors: Stanton J. Rowe, Newport Coast, CA (US); Gregory Bak-Boychuk, San Clemente, CA (US); Juan Valencia, Fullerton, CA (US); Robert S. Schwartz, Inver Grove Heights, MN (US)

(21) Appl. No.: 16/178,343

(22) Filed: Nov. 1, 2018

Related U.S. Application Data
(60) Provisional application No. 62/585,623, filed on Nov. 14, 2017.

(51) Int. Cl. A61M 1/12; A61M 1/10; A61F 2/24

(52) U.S. Cl. CPC A61M 1/122; A61M 1/125; A61F 7/10; A61F 2/2481; A61M 1/1008

(57) ABSTRACT

An organ restraint device includes a first end portion including a fluid inlet channel, a second end portion including a fluid outlet channel, a medial body portion coupled between the first end portion and the second end portion. The medial body portion includes one or more fluid channels that couple the fluid inlet channel to the fluid outlet channel and a plurality of tension adjustment apertures

### RELATED APPLICATION

This application claims priority to U.S. Provisional Application No. 62/585,623, filed Nov. 14, 2017, and entitled ORGAN RESTRAINT FOR ATRIAL FIBRILLATION PREVENTION, the disclosure of which is hereby incorporated by reference in its entirety.

### BACKGROUND

### Field

The present disclosure generally relates to the field of vascular surgery, such as cardiac surgery.

### Description of Related Art

Patients of cardiac surgery and other vascular operations can develop certain inflammation conditions and/or atrial fibrillation post-operatively due to various conditions and/or factors. Atrial fibrillation is associated with certain health complications, including increased patient mortality, and therefore prevention and/or treatment of atrial fibrillation during surgery and/or post-operatively can improve patient health.

### SUMMARY

In some implementations, the present disclosure relates to an organ restraint device comprising a first end portion including a fluid inlet channel, a second end portion including a fluid outlet channel, and a medial body portion coupled between the first end portion and the second end portion. The medial body portion comprises one or more fluid channels that couple the fluid inlet channel to the fluid outlet channel, and a plurality of tension adjustment apertures.

In certain embodiments, the fluid inlet channel, the fluid outlet channel, and the one or more fluid channels are configured to allow cooling fluid to be introduced into the organ restrain device through the fluid inlet channel, passed through the one or more fluid channels of the medial body portion, and expelled out of the organ restraint device through the fluid outlet channel The medial body portion comprises an elongate band, and/or one or more transversely-projecting contours. For example, the one or more transversely-projecting contours can comprise first and second longitudinally-spaced transversely-projecting contours, with a recessed groove therebetween. The first and second transversely-projecting contours can be shaped for contacting left and right atria, respectively, of a heart.

The medial body portion may further comprise first and second longitudinally-aligned transversely-projecting contours that project in opposing directions and form a left atrium contact pad, and third and fourth longitudinally-aligned transversely-projecting contours that project in opposing directions and form a right atrium contact pad, wherein the left atrium contact pad is longitudinally offset from the right atrium contact pad.

In certain embodiments, the medial body portion comprises flexible polymer. The one or more fluid channels of the medial body portion may comprise first and second parallel fluid channels that branch from one or more of the fluid inlet channel and the fluid outlet channel. In certain embodiments, one or more of the first end portion and the second end portion comprise a fluid tube connection fitting.

In some implementations, the present disclosure relates to a method of treating atria of a heart to prevent atrial fibrillation. The method may comprise inserting a first end of an organ restraint device into a chest cavity of a patient, passing the first end of the organ restraint device around a posterior side of a heart of the patient, wrapping the organ restrain device over one or more of a right atrium and a left atrium of the heart, threading a tension adjustment line through one or more first apertures associated with a first longitudinal half of the organ restrain device, threading the tension adjustment line through one or more second apertures associated with a second longitudinal half of the organ restraint device, drawing the one or more first apertures towards the one or more second apertures by pulling on the tension adjustment line, inserting cooling fluid into one of the first end and a second end of the organ restraint device, and expelling the cooling fluid from another of the first end and the second end.

The method may further comprise applying pressure to the one or more of the right atrium and the left atrium of the heart using the organ restraint device. For example, applying pressure may restrain one or more of the left and right atria from expanding beyond 5 mm in diameter.

In certain embodiments, the method further comprises inserting the tension adjustment line into a first end of a compression tube. For example, the method may comprise withdrawing the tension adjustment line from a second end of the compression tube, wherein said pulling on the tension adjustment line involves pulling the tension adjustment line through the compression tube. The method may further comprise locking the tension adjustment line externally to the patient. For example, locking the tension adjustment line may be performed using a locking mechanism coupled to the tension adjustment line and disposed externally to the patient.

Passing the first end of the organ restraint device around the posterior side of the heart may involve passing the first end through a pocket between an aorta or pulmonary artery of the patient and one or more atria of the heart. Furthermore, drawing the one or more first apertures towards the one or more second apertures may at least partially prevent stretching of one or more atria of the heart due to fluid overload associated with a surgical operation. In certain embodiments, inserting and expelling the cooling fluid reduces inflammation of one or more of the left and right atria to prevent atrial fibrillation. The method may further comprise controlling a temperature of the cooling fluid to provide a desired therapeutic effect. The method may further comprise nesting the first and second ends of the organ restraint device in one or more chest drainage tubes.

In some implementations, the present disclosure relates to a method of removing an organ restraint device from a chest cavity of a patient. The method comprises unlocking a tension adjustment line threaded through one or more apertures of an organ restraint device wrapped around one or more of a right atrium and a left atrium of a heart of a patient, withdrawing the tension adjustment line from a chest cavity of the patient, evacuating fluid from the restrain device, and withdrawing the organ restraint device from the chest cavity of the patient by pulling on a first end of the organ restraint device to thereby draw a second end of the organ restraint device around a posterior side of the heart. The method may be performed while the chest cavity of the patient is closed.

Withdrawing the tension adjustment line from the chest cavity may involve pulling the tension adjustment line through a first chest tube implanted in the patient. For example, withdrawing the organ restraint device from the chest cavity may involve pulling the organ restraint device through a second chest tube implanted in the patient. In certain embodiments, the method is performed between three and five days after a surgical operation involving the patient. Unlocking the tension adjustment line may involve disengaging a locking mechanism external to the patient. For example, the locking mechanism may comprise a hemostat.

In some implementations, the present disclosure relates to an atria restraint band comprising a first distal end portion, a second distal end portion, and a first atrium contact portion positioned along a longitudinal dimension of the atria restraint band, the first atrium contact portion comprising a first protrusion in a first transverse direction, and a second protrusion in a second transverse direction opposite the first transverse direction, the second protrusion being aligned longitudinally with the first protrusion. The atria restrain band further comprises a second atrium contact portion positioned along the longitudinal dimension of the atria restraint band, the second atrium contact portion comprising a third protrusion in the first transverse direction, and a fourth protrusion in the second transverse direction, the third protrusion being aligned longitudinally with the fourth protrusion. The atria restrain band further comprises a transversely-recessed groove positioned between the first and second atrium contact portions along the longitudinal dimension of the atria restraint band.

In certain embodiments, the first and second atrium contact portions are shaped to cover a majority of a surface area of an atrium of a heart. The transversely-recessed groove may be shaped to accommodate the presence of a blood vessel to reduce deformation or displacement thereof when the atria restrain band is implanted in proximity to the blood vessel. For example, the blood vessel is a superior vena cava, a pulmonary artery, or an aorta. In certain embodiments, the atria restraint band further comprises an edge support wire associated with an edge portion of the atria restraint band.

### BRIEF DESCRIPTION OF THE DRAWINGS

Various embodiments are depicted in the accompanying drawings for illustrative purposes, and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 provides an example cross-sectional view of a human heart.

FIG. 2 illustrates an example cross-sectional representation of a heart experiencing atrial fibrillation.

FIG. 3 illustrates a perspective view of an organ restraint device in accordance with one or more embodiments.

FIG. 4A illustrates an unwrapped view of an organ restraint band in accordance with one or more embodiments.

FIG. 4B illustrates an unwrapped view of an atria restraint band in accordance with one or more embodiments.

FIG. 5 illustrates a perspective view an organ restraint band wrapped at least partially around the atria of a heart in accordance with one or more embodiments.

FIG. 6 illustrates an organ restraint band in accordance with one or more embodiments.

FIG. 7 illustrates a perspective view of an example patient in a post-operative state.

FIG. 8 illustrates a cutaway view of an organ restraint and therapy system in accordance with one or more embodiments.

FIG. 9 illustrates a top-down view of an organ restraint and therapy system in accordance with one or more embodiments.

FIG. 10 illustrates an inverted side view of a heart having an atria restraint band associated therewith in accordance with one or more embodiments.

FIG. 11A illustrates a posterior surface view of a heart having an atria restraint band associated therewith in accordance with one or more embodiments.

FIG. 11B illustrates a posterior surface view of a heart having an atria restraint band associated therewith in accordance with one or more embodiments.

FIG. 12 illustrates an atria restraint band passed behind a heart in accordance with one or more embodiments.

FIG. 13 provides a view of the heart illustrated in FIG. 12 having a restraint band wrapped over the atria of the heart in accordance with one or more embodiments.

FIG. 14A illustrates a close-up view of a portion of the diagram of FIG. 13 showing restraint band threading in accordance with one or more embodiments.

FIG. 14B illustrates a close-up view of a portion of the diagram of FIG. 13 showing a distal end portion of a compression tube in accordance with one or more embodiments.

FIG. 15 illustrates a stage of a process for implanting and securing a restraint band in accordance with one or more embodiments.

FIGS. 16-18 illustrate stages of a restraint band removal process according to one or more embodiments disclosed.

FIG. 19 illustrates a perspective view of a restraint band draped over the atria of a heart in accordance with one or more embodiments.

FIG. 20 illustrates a restraint device having a ventricular restraint pouch and atrial restraint straps in accordance with one or more embodiments.

FIG. 21 illustrates a restraint device having a ventricular restraint pouch and atrial restraint straps in accordance with one or more embodiments.

FIG. 22 illustrates a restraint device having a ventricular restraint pouch and atrial restraint straps in accordance with one or more embodiments.

### DETAILED DESCRIPTION

The headings provided herein are for convenience only and do not necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Terminology

Certain standard anatomical terms of location are used herein to refer to the anatomy of animals, and namely humans, with respect to the preferred embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. Spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Furthermore, references may be made herein to certain anatomical planes, such as the sagittal plane, or median plane, or longitudinal plane, referring to a plane parallel to the sagittal suture, and/or other sagittal planes (i.e., parasagittal planes) parallel thereto. In addition, “frontal plane,” or “coronal plane,” may refer to an X-Y plane that is perpendicular to the ground when standing, which divides the body into back and front, or posterior and anterior, portions. Furthermore, a “transverse plane,” or “cross-sectional plane,” or horizontal plane, may refer to an X-Z plane that is parallel to the ground when standing, and that divides the body in upper and lower portions, such as superior and inferior. A “longitudinal plane” may refer to any plane perpendicular to the transverse plane. Furthermore, various axes may be described, such as a longitudinal axis, which may refer to an axis that is directed towards head of a human in the cranial direction and/or directed towards inferior of a human in caudal direction. A left-right or horizontal axis, which may refer to an axis that is directed towards the left-hand side and/or right-hand side of a patient. An anteroposterior axis which may refer to an axis that is directed towards the belly of a human in the anterior direction and/or directed towards the back of a human in the posterior direction.

### Overview

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.). The contraction of the various heart muscles may be prompted by signals generated by the electrical system of the heart, which is discussed in detail below. Certain embodiments disclosed herein relate to conditions of the heart, such as atrial fibrillation and/or complications or solutions associated therewith. However, embodiments of the present disclosure relate more generally to any health complications relating to tissue or organ inflammation and/or fluid overload in a patient, such as may result post-operatively after any surgery, including surgeries involving fluid supplementation. That is, restraint of tissue/organs as described herein may be implemented to prevent or reduce incidences of tissue/organ inflammation, expansion, dilation, stretching, or other undesirable alteration thereof.

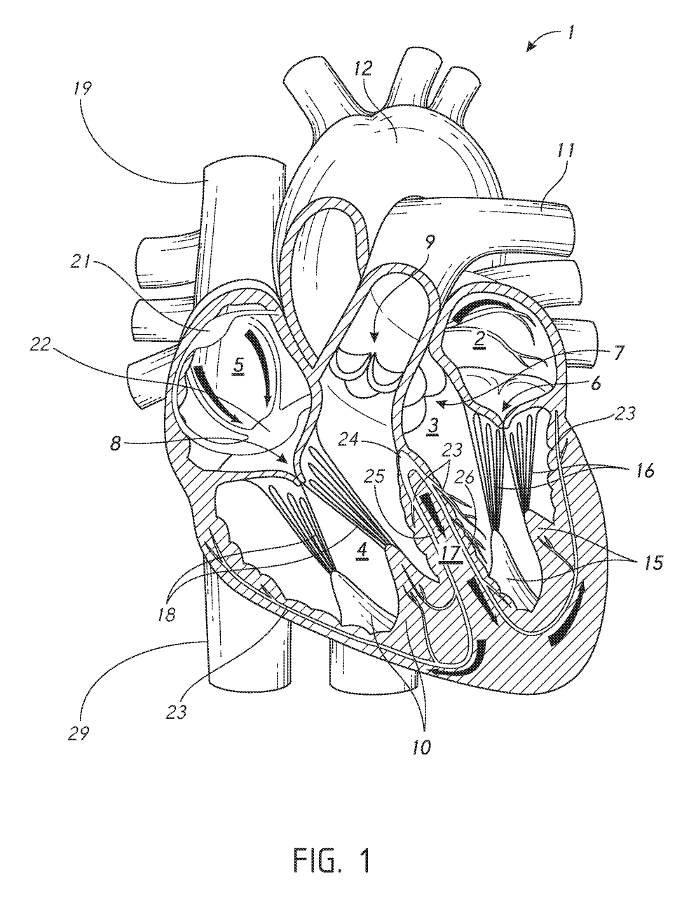

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery 11 and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size and position of the leaflets or cusps may be such that when the heart contracts, the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

The atrioventricular (i.e., mitral and tricuspid) heart valves may further comprise a collection of chordae tendineae (16, 18) and papillary muscles (10, 15) for securing the leaflets of the respective valves to promote and/or facilitate proper coaptation of the valve leaflets and prevent prolapse thereof. The papillary muscles (10, 15), for example, may generally comprise finger-like projections from the ventricle wall. With respect to the mitral valve 6, a normal mitral valve may comprise two leaflets (anterior and posterior) and two corresponding papillary muscles 15. When the left ventricle 3 contracts, the intraventricular pressure forces the valve to close, while the chordae tendineae 16 keep the leaflets coapting together and prevent the valve from opening in the wrong direction, thereby preventing blood to flow back to the left atrium 2. With respect to the tricuspid valve 8, the normal tricuspid valve may comprise three leaflets (two shown in FIG. 1) and three corresponding papillary muscles 10 (two shown in FIG. 1). The leaflets of the tricuspid valve may be referred to as the anterior, posterior and septal leaflets, respectively. The valve leaflets are connected to the papillary muscles by the chordae tendineae 17, which are disposed in the right ventricle 4 along with the papillary muscles 10. The right ventricular papillary muscles 10 originate in the right ventricle wall, and attach to the anterior, posterior and septal leaflets of the tricuspid valve, respectively, via the chordae tendineae 17.

### Post-Operative Inflammation

Inflammation generally involves a biological response of body tissues to harmful stimuli, such as pathogens, damaged cells, or irritants. The response is protective in nature and generally involves immune cells, blood vessels, and/or molecular mediators. Inflammation can manifest as sensations of heat and/or pain, redness, swelling, and/or loss of function in the inflamed tissue/organ. Acute inflammation can result post-operatively as a complication of a surgical operation, such as a cardiac surgery. For example, contusion or contamination occurring during surgery can cause inflammation to develop. With respect to cardiac inflammation, heart failure and/or death can result in certain cases if adequate treatment is not implemented. Therefore, prevention of inflammation in connection with surgical operations and/or reduction or treatment of inflammation soon after or during a surgical operation can improve patient health. Embodiments of the present disclosure advantageously provide devices, systems, and methods for preventing and/or treating inflammation during and after surgical operations.

### Fluid Overload

Fluid overload or volume overload, which is referred to as hypervolemia, is a medical condition in which the vasculature contains too much fluid. Fluid-overload conditions can arise in connection with various types of surgical operations, including cardiac surgery. For example, fluid management through fluid infusion may be necessary or desirable in order to maintain adequate cardiac output, systemic blood pressure, and/or renal perfusion during or in connection with a surgical operation. Example settings in which fluid overload may develop include the administration of excessive fluid and sodium due to intravenous (IV) or fluids during surgical operations, such as atrial fibrillation ablation, valve repair or replacement, or other cardio/thoracic procedures, or fluid remobilization procedures associated with burn or trauma treatment.

Fluid overload can correlate with mortality in certain categories of patients. In order to restore or maintain desired fluid levels, it may be necessary or desirable to determine present volume status. According to some practices, fluid overload recognition and assessment involves strict documentation of fluid intakes and outputs. However, accuracy is fluid intake/output tracking can be difficult to achieve over time, and there are a wide variety of methods utilized to evaluate, review, and utilize fluid tracking data. Furthermore, errors in volume status determination can result in a lack of essential treatment or unnecessary fluid administration, either of which can present serious health risks.

As described herein, fluid overload associated with fluid administration of fluid in association with a surgical operation can result in post-operative onset of atrial fibrillation. Furthermore, fluid overload conditions can cause or be associated with various other conditions, including pulmonary edema, cardiac failure, delayed recovery, tissue breakdown, and/or at least partially impaired function of bowels or other organs. Therefore, the evaluation of volume status can be important before, during, and/or after a surgical operation, such as cardia surgery. Once identified, fluid overload may be treated in a variety of ways, including cessation or reduction of fluid administration, administration of diuretics, and/or fluid/letting.

For at least the reasons outlined above, determination/detection of fluid overload conditions can be critical or important to prevention or treatment of various adverse health conditions. However, the lack of available volume overload sensors that conveniently and accurately measure or indicate fluid overload can be problematic. Embodiments of the present disclosure provide improved systems, devices, and methods for determining/detecting a fluid overload condition by monitoring tissue stretching in fluid-containing organs or tissue. For example, tissue stretching in an atrium (or ventricle) of a heart, as described in detail herein, can indicate a fluid overload, or impending fluid overload, condition. The embodiments of the present disclosure advantageously provide removable devices/systems for measuring tissue stretching associated with fluid overload in a relatively convenient manner compared to pressure measurement fluid tracking using, for example, peripherally-inserted central catheter (PICC or PIC line), or other known mechanism for tracking of fluid pressure or other characteristic(s). Certain embodiments of the present disclosure provide improvements over other patient monitoring solutions by providing systems, devices, and methods for directly measuring organ or tissue stretching, wherein it is not necessary to infer tissue stretching from echo or x-ray imaging. Direct tissue-measuring in accordance with embodiments of the present disclosure may be used to measure atrial tissue stretching, or stretching of other organs or tissue, including but not limited to gestational stretch measurement of uterine tissue or other pregnancy-related stretching, prostate stretching/enlargement, liver tissue stretching, colon stretching/enlargement, or other tissue/organ.

### Cardiac Electrical System

The electrical system of the heart generally controls the events associated with the pumping of blood by the heart. With further reference to FIG. 1, the heart 1 comprises different types of cells, namely cardiac muscle cells (also known as cardiomyocytes or myocardiocytes) and cardiac pacemaker cells. For example, the atria (2, 5) and ventricles (3, 4) comprise cardiomyocytes, which are the muscle cells that make up the cardiac muscle. The cardiac muscle cells are generally configured to shorten and lengthen their fibers and provide desirable elasticity to allow for stretching. Each myocardial cell contains myofibrils, which are specialized organelles consisting of long chains of sarcomeres, the fundamental contractile units of muscle cells.

The electrical system of the heart utilizes the cardiac pacemaker cells, which are generally configured to carry electrical impulses that drive the beating of the heart 1. The cardiac pacemaker cells serve to generate and send out electrical impulses, and to transfer electrical impulses cell-to-cell along electrical conduction paths. The cardiac pacemaker cells further may also receive and respond to electrical impulses from the brain. The cells of the heart are connected by cellular bridges, which comprise relatively porous junctions called intercalated discs that form junctions between the cells. The cellular bridges permit sodium, potassium and calcium to easily diffuse from cell-to-cell, allowing for depolarization and repolarization in the myocardium such that the heart muscle can act as a single coordinated unit.

The electrical system of the heart comprises the sinoatrial (SA) node 21, which is located in the right atrium 5 of the heart 1, the atrioventricular (AV) node 22, which is located on the interatrial septum in proximity to the tricuspid valve 8, and the His-Purkinje system 23, which is located along the walls of the left 3 and right 4 ventricles.

A heartbeat represents a single cycle in which the heart's chambers relax and contract to pump blood. As described above, this cycle includes the opening and closing of the inlet and outlet valves of the right and left ventricles of the heart. Each beat of the heart is generally set in motion by an electrical signal generated and propagated by the heart's electrical system. In a normal, healthy heart, each beat begins with a signal from the SA node 21. This signal is generated as the vena cavae (19, 29) fill the right atrium 5 with blood, and spreads across the cells of the right 5 and left 2 atria. The flow of electrical signals is represented by the illustrated shaded arrows in FIG. 1. The electrical signal from the SA node 21 causes the atria to contract, which pushes blood through the open mitral 6 and tricuspid 8 valves from the atria into the left 3 and right 4 ventricles, respectively.

The electrical signal arrives at the AV node 22 near the ventricles, where it may slow for an instant to allow the right 4 and left 3 ventricles to fill with blood. The signal is then released and moves along a pathway called the bundle of His 24, which is located in the walls of the ventricles. From the bundle of His 24, the signal fibers divide into left 26 and right 25 bundle branches through the Purkinje fibers 23. These fibers connect directly to the cells in the walls of the left 3 and right 4 ventricles. The electrical signal spreads across the cells of the ventricle walls, causing both ventricles to contract. Generally, the left ventricle may contract an instant before the right ventricle. Contraction of the right ventricle 4 pushes blood through the pulmonary valve 9 to the lungs (not shown), while contraction of the left ventricle 3 pushes blood through the aortic valve 6 to the rest of the body. As the electrical signal passes, the walls of the ventricles relax and await the next signal.

### Atrial Fibrillation

FIG. 1, as described above, illustrates a normal electrical flow, resulting in a regular heart rhythm that may be associated with a generally healthy heart. However, in certain patients or individuals, various conditions and/or events can result in compromised electrical flow, causing the development and/or occurrence of an abnormal heart rhythm. For example, atrial fibrillation is a condition associated with abnormal electrical flow and/or heart rhythm characterized by relatively rapid and irregular beating of the atria.

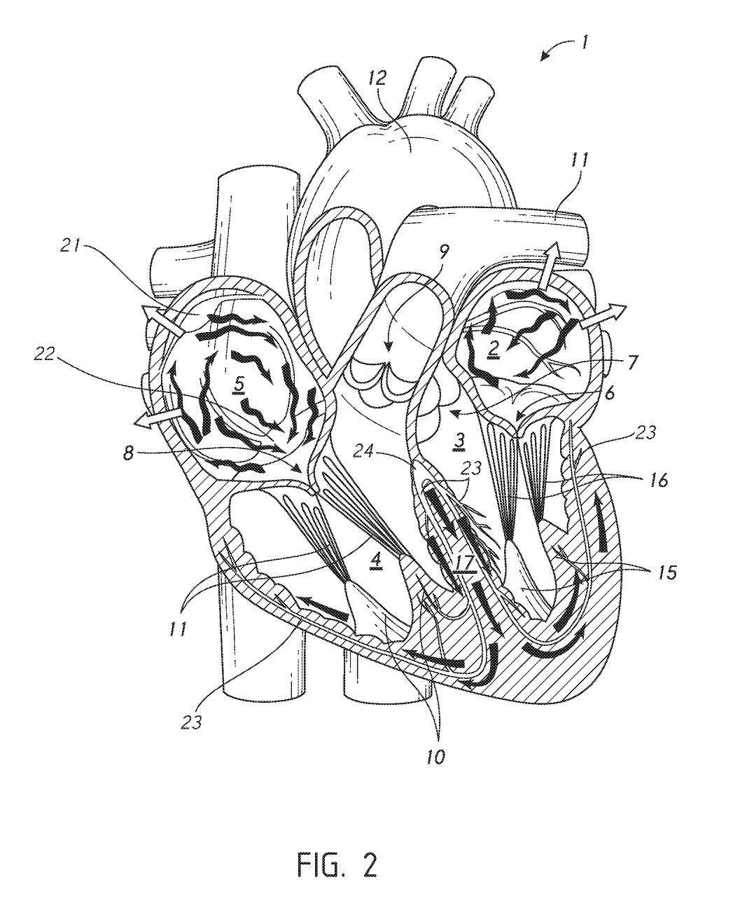

FIG. 2 illustrates an example cross-sectional representation of the heart 1 of FIG. 1 experiencing atrial fibrillation. When atrial fibrillation occurs, the normal regular electrical impulses generated by the sinoatrial (SA) node 21 in the right atrium 5 may become overwhelmed by disorganized electrical impulses, which may lead to irregular conduction of ventricular impulses that generate the heartbeat. The illustrated shaded arrows represent the erratic electrical impulses that can be associated with atrial fibrillation. Atrial fibrillation generally originates in the right atrium 5, that where conduction path disturbances begin.

Various pathologic developments can lead to, or be associated with, atrial fibrillation. For example, progressive fibrosis of the atria may contribute at least in part to atrial fibrillation. The formation of fibrous tissue associated with fibrosis can disrupt or otherwise affect the electrical pathways of the cardiac electrical system due to interstitial expansion associated with tissue fibrosis. In addition to fibrosis in the muscle mass of the atria, fibrosis may also occur in the sinoatrial node 21 and/or atrioventricular node 22, which may lead to atrial fibrillation.

Fibrosis of the atria may be due to atrial dilation, or stretch, in some cases. Dilation of the atria can be due to a rise in the pressure within the heart, which may be caused by fluid overload, or may be due to a structural abnormality in the heart, such as valvular heart disease (e.g., mitral stenosis, mitral regurgitation, or tricuspid regurgitation), hypertension, congestive heart failure, or another condition. Dilation of the atria can lead to the activation of the renin aldosterone angiotensin system (RAAS), and subsequent increase in matrix metalloproteinases and disintegrin, which can lead to atrial remodeling and fibrosis and/or loss of atrial muscle mass.

In addition to atrial dilation, inflammation in the heart can cause fibrosis of the atria. For example, inflammation may be due to injury associated with a cardiac surgery, such as a valve repair operation, or the like. Alternatively, inflammation may be caused by sarcoidosis, autoimmune disorders, or other condition. Other cardiovascular factors that may be associated with the development of atrial fibrillation include high blood pressure, coronary artery disease, mitral stenosis (e.g., due to rheumatic heart disease or mitral valve prolapse), mitral regurgitation, hypertrophic cardiomyopathy (HCM), pericarditis, and congenital heart disease. Additionally, lung diseases (such as pneumonia, lung cancer, pulmonary embolism, and sarcoidosis) may contribute to the development of atrial fibrillation in some patients.

### Development of Post-Operative Atrial Fibrillation

In addition to the various physiological conditions described above that may contribute to atrial fibrillation, in some situations, atrial fibrillation may be developed in connection with a vascular operation, such post-operatively in the days following a vascular operation. Various factors may bear on the likelihood of a patient developing post-operative atrial fibrillation, such as age, medical history (e.g., history of atrial fibrillation, chronic obstructive pulmonary disease (COPD)), concurrent valve surgery, withdrawal of post-operative treatment (e.g., beta-adrenergic blocking agents (i.e., beta blocker), angiotensin converting enzyme inhibitors (ACE inhibitor)), beta-blocker treatment (e.g., pre-operative and/or post-operative), ACE inhibitor treatment (e.g., pre-operative and/or post-operative), and/or other factors. Generally, for patients that experience post-operative atrial fibrillation, the onset of atrial fibrillation may occur approximately 2-3 days after surgery.

Atrial dilation/stretching may be considered a primary variable associated with post-operative atrial fibrillation. In some situations, occurrence of post-operative atrial fibrillation may follow, at least in part, the following progression: First, the patient undergoes a surgical procedure, such as a vascular surgical operation (e.g., cardiac surgery). In connection with the operation, the patient may be subject to drug and/or fluid management. For example, the patient may receive post-surgery intravenous (IV) fluid loading and/or diuretic/drug volume management. Such treatment may result in fluid overload, which may lead to atrial stretching due to increased pressure in one or more atria. Atrial stretching may occur over a 1-2-day period, or longer, resulting in dilation of one or both of the atria. Fibrotic atrial tissue may form in connection with atrial stretching. Atrial stretching and/or fibrotic atrial tissue formation may result in an increased incidence of post-operative atrial fibrillation (e.g., 30-40% increased incidence of post-operative atrial fibrillation). In addition, inflammation associated with surgical operations can contribute the onset of post-operative atrial fibrillation, and reduced inflammation may generally correlate to a reduced risk of atrial fibrillation.

Post-operative atrial fibrillation is generally associated with increased patient morbidity, as well as economic burden. For example, post-operative atrial fibrillation is generally associated with increased incidence of congestive heart failure, increased hemodynamic instability, increase renal insufficiency, increased repeat hospitalizations, increased risk of stroke, and increase in hospital mortality and 6-month mortality. Post-operative atrial fibrillation also represents a systemic burden, wherein intensive care unit (ICU) stay, hospital length of stay, hospital charges, and rates of discharge to extended care facilities are increased as a result of post-operative atrial fibrillation.

Furthermore, because an initial incidence of atrial fibrillation generally results in recurring, progressively more severe, episodes of atrial fibrillation in a patient, the consequences of allowing atrial fibrillation to develop post-operatively can be considered particularly severe for a given patient. For example, a given patient may initially experience intermittent/sporadic episodes of atrial fibrillation as a result of post-operative atrial dilation and/or inflammation, with recurring episodes progressively increasing in frequency and/or severity.

### Prevention of Post-Operative Atrial Stretch and Inflammation

The development of atrial fibrillation post-operatively can have a serious negative impact on patient quality of life. As discussed above, atrial stretch and inflammation may represent root causes of post-operative atrial fibrillation in some situations. Therefore, by reducing or restricting atrial stretch and/or inflammation during vascular surgery, or over a period of time thereafter, incidences of post-operative atrial fibrillation can be reduced. The majority of post-operative atrial fibrillation instances may occur within the first two days after surgery, and therefore, prevention of post-operative atrial stretch and/or inflammation may be particularly significant during the initial days after surgery.

Generally, atrial diameter expansion of greater than 5 mm may be correlated with chronic atrial fibrillation in some cases. Furthermore, increase in atrial circumference of greater than 10%, and/or increase in atrial volume of greater than 8.5 mL may be associated with chronic atrial fibrillation. Therefore, embodiments disclosed herein may be designed to limit or restrict atrial stretch to prevent expansion of atrial diameter by 5 mm or more, increase in circumferential stretch by greater than 10%, and/or increase in atrial volume by 8.5 mL or more in order to reduce incidences of atrial fibrillation. With regard to fluid overload, in some situations, the introduction of around 1.5 additional liters of fluid to a patient's vascular system may be correlated with increased rates of atrial fibrillation. Generally, the greater the amount of fluid added, the greater the amount of atrial stress that may be experienced by the patient.

In some implementations, the present disclosure provides a means for restricting atrial stretching in either or both of the left and right atria, and/or the reduction of inflammation associated with the atria, for a post-operative period after a surgical procedure, thereby reducing the likelihood of onset of post-operative atrial fibrillation. For example, embodiments disclosed herein may be suitable for restricting atrial stretching and/or reducing inflammation for a period of up to five days after a surgical procedure. In some implementations, a post-operative atrial fibrillation prevention device may be implanted or applied at the time of surgery, but may advantageously be removed at a later time. For example, in some embodiments, an atrial fibrillation prevention device may be removed at or about the time that chest drainage tubes associated with a surgical operation are removed, which may correspond with a time period approximately five days after completion of the surgery, or other time period.

### Organ/Atria Restraint Bands

As described in detail above, fluid volume overload in the vascular system of a patient, and in particular within the atria, can cause an increase in atrial pressure. When exposed to elevated atrial pressures, atrial tissue may be inclined to stretch over time. Various mechanisms, devices, and processes are disclosed herein for at least partially restraining the left and/or right atrium from stretching to thereby reduce the risk of post-operative atrial fibrillation. Atrial restraint devices and methods disclosed herein may advantageously at least partially restrict the expansion or stretching of atrial tissue, while allowing for desirable expansion of the atria in order to accommodate the proper contraction and expansion of the atria typically associated with each heartbeat cycle. For example, that diameter of an atrium may change by approximately 2 mm per beat for a healthy heart. Therefore, in some implementations, devices and methods for restraining atrial stretch according to the present disclosure may advantageously accommodate approximately 2 mm per beat of diameter change of the atria, but at least partially limit stretching beyond that.

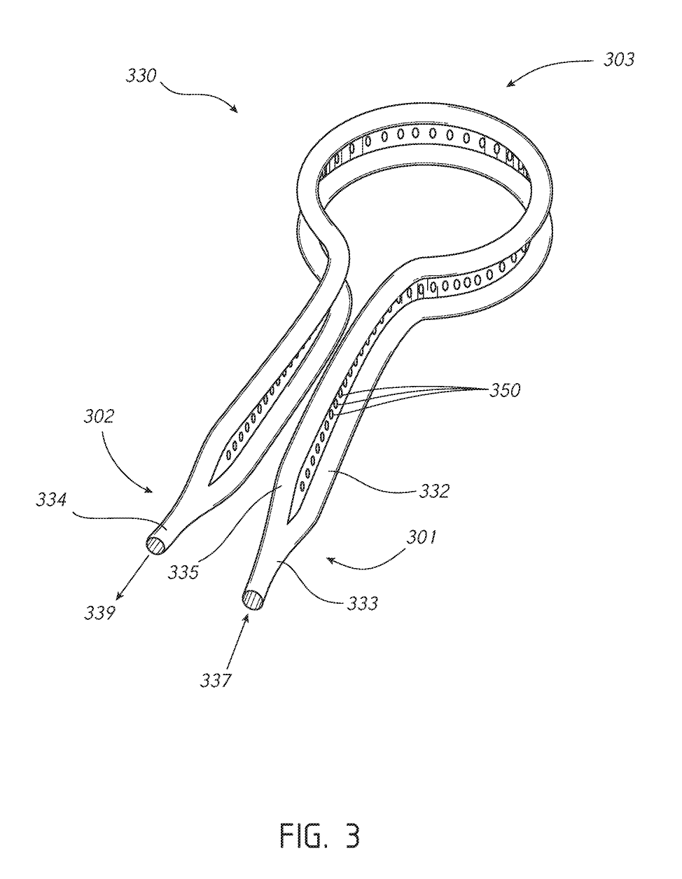

FIG. 3 illustrates a perspective view of an organ restraint device 330, referred to below for convenience as an atria restraint band, in accordance with one or more embodiments disclosed herein. The atria restraint band 330 may be a removable organ restraint band configured to restrain one or more of the atria of a heart. In some implementations, the band 330 is also configured with temperature-control features, which may help to reduce inflammation and/or otherwise reduce the risk of atrial fibrillation. The atria restraint band 330 may be configured to be draped or wrapped around one or more of the atria of the heart, to thereby provide pressure thereto to restrict outward expansion of the atria. Although embodiments are disclosed herein in the context of restraint of the atria of the heart, it should be understood that the principles disclosed herein may be applicable to restraint of other organs, or portions thereof. Furthermore, although certain embodiments of organ restraint devices are referred to herein as “bands,” organ (e.g., atria) restraint devices in accordance with the present disclosure may have any suitable or desirable shape or configuration.

The band 330 may include first and second distal, or end, portions 301, 302, and a medial body portion 303 configured to wrap around and/or physically contact an organ or portion thereof to be restrained. In certain embodiments, the band 330 comprises flexible polymer film, or other flexible biocompatible material. In some implementations, the band 330 may be configured to be at least partially filled with cooling fluid (not shown) in order to provide a cooling effect for the band 330 with respect to tissue in contact or proximity therewith. For example, in some implementations, the band 330 comprises one or more fluid circulation pathways, or channels, such as the illustrated fluid channels 332, 333, 334, 335. One or both of the fluid channels 333, 334 may serve as a fluid inlet channel for introducing cooling fluid into the device 330, and one or both of the fluid channels 333, 334 may serve as a fluid outlet channel for expelling cooling fluid from the band 330. One or more fluid channels, such as the parallel fluid channels 332, 335, may branch off from the fluid inlet/outlet channels (333, 334) and pass fluid therebetween along a longitudinal length of the band 330 between the end portions 301, 302.

The use of cooling fluid with the restraint band 330 may advantageously serve to reduce inflammation of the tissue contacting or in physical proximity to the restraint band 330, which may promote healing of the cooled tissue. For example, where a surgical wound is present, such as in one or more of the atria of the heart, the cooling functionality of the band 330 may advantageously promote healing and/or reduce inflammation associated with the wound. Furthermore, where fluid is introduced and/or maintained within one or more pathways or channels of the band 330, such fluid may be utilized to provide pressure within the band, which may advantageously be used to introduce and/or subject the restrained organ or tissue (e.g., atrium) to increased or desirable pressure for restraint purposes. Furthermore, the pressure of the fluid in the band 330 may also provide the band desirable structure and/or rigidity in one or more portions thereof, and may further provide an adjustment mechanism for the band for selectively providing desirable levels of pressure and/or providing pressure in desired locations or areas of the band 330 and/or restrained organ.

As described herein, inflammation of the atrium after cardiac surgery may be associated with inhomogeneity of atrial conduction and/or atrial fibrillation. In some cases, topical or surface application of anti-inflammatory substances and/or cooling means may reduce the risk of atrial fibrillation occurring after a surgical operation. Cooling fluid may be maintained within the band, and/or circulated therethrough, in order to provide desired cooling functionality of the band 330. For example, cooling fluid may be introduced into a distal fluid inlet channel or pathway 333, as represented by the arrow 337. Fluid may be permitted to pass through a length of the band 330 and exit a second distal end channel or pathway 334. In the illustrated embodiment, the fluid introduced into the band 330 can be divided between the first and second fluid parallel channels or pathways 335, 332, which may run along a length of the band 330. Although two parallel fluid channels or pathways are illustrated in the band 330, it should be understood that the band 330 may comprise any number or configuration of fluid channels or pathways, wherein such channels or pathways may have any suitable or desirable length, shape, and/or diameter or other dimension. Although certain embodiments are disclosed herein in the context of liquid cooling fluid, it should be understood that gaseous cooling fluid may be used in organ restraint bands disclosed herein according to one or more embodiments.

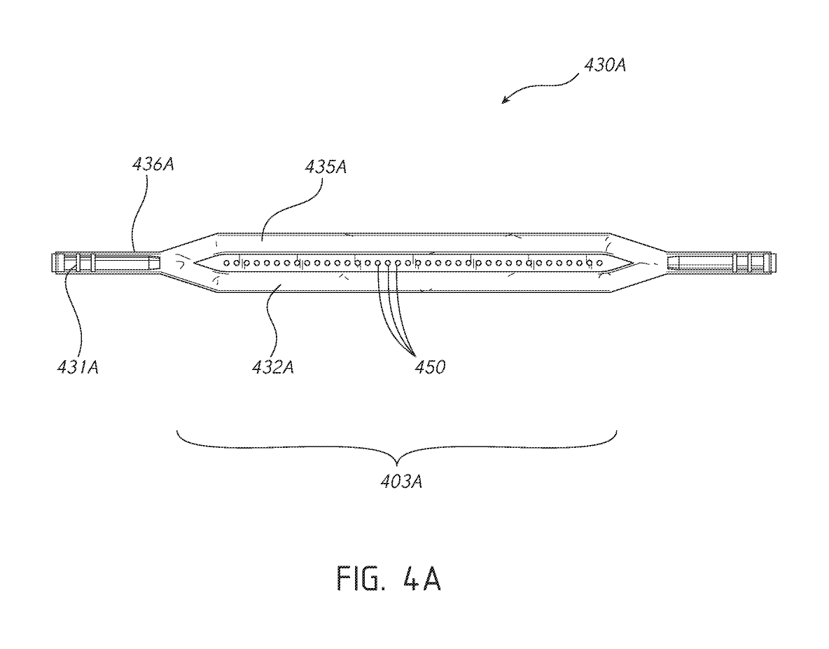

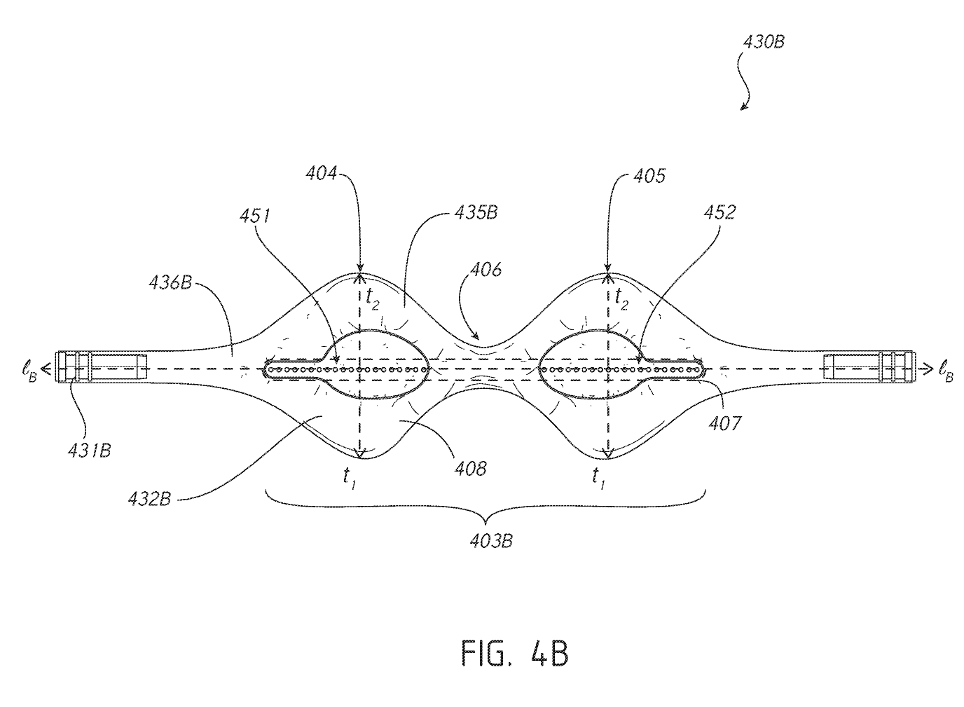

FIG. 4A illustrates an unwrapped view of an organ restraint band 430 in accordance with one or more embodiments. As described, in some implementations, the band 430A is a removable atria restraint band with fluid passages 432A, 435A, 436A configured and dimensioned to contain and/or circulate cooling fluid. The band 430A may be constructed at least partially from polymer film, such as at least partially transparent or clear polymer film. In certain embodiments, the film or other material of which the band 430A is comprised may be fluid sealed, such as through a heat-sealing process, or the like. In certain embodiments, the band 430A includes a fluid tube connection fitting 431 at one or more distal ends of the band 430A, which may provide an interface for coupling a fluid source, or otherwise introducing fluid into one or more channels of the band 430A. The fluid channels 432A, 435A may extend across a medial body portion 403A of the band 430A.

FIG. 4B illustrates an unwrapped view of an alternative embodiment of an atria restraint band 430B in accordance with one or more embodiments. Unlike the illustrated band 430A of FIG. 4A, the band 430B illustrated in FIG. 4B may be configured with one or more transversely-projecting contours or bulges along a medial portion 403B of the band 430. Such projecting contours (e.g., 404, 405) may generally jut out from a longitudinal axis of the band in a generally-transverse direction tₗ, t₂. The bulges, or protrusions, 404, 405 may provide increased surface area for the band 430B in one or more regions thereof, and may be designed to increase the surface area coverage of the atria or other organ to be restrained. For example, opposite-facing (i.e., projecting in opposite transverse directions) contours (e.g., contours 404, 408) may collectively provide an atrium contact pad restraining one of the left and right atria of the heart. The opposite-facing transverse projections (e.g., contours 404, 408) may be aligned longitudinally with respect to the longitudinal axis l_{B} of the band 430B. The contours of the band 430B may further form one or more transverse grooves/depressions, or notches, 406, which may allow the band 430B to be wrapped around the heart or other organ, while accommodating the natural disposition of certain proximate anatomy, such as a blood vessel. For example, the recessed groove 406 may accommodate the presence of the aorta and/or superior vena cava, pulmonary veins/arteries, or other blood vessels.

With respect to the dimensions and shape of the transversely projecting contours/protrusions 404, 405, the shape of such protrusions be may help even-out the pressure applied by the band 430B over a greater surface area of the atria, to thereby avoid strain from the portions of the band that are aligned with the atria from being concentrated along transversely central strip 407 of the band 430B. For example, without contours or other shapes designed to fit the shape of the atria, the pressure on the atria may not be desirably even over the surface of the atria, but rather may be concentrated along the transversely central band/line 407 of the restraint band 430B.

The shape of the illustrated band 430B may help prevent pinching or choking-off of the superior vena cava, or other blood vessel, while still allowing for restraint of the atria by a wrapped band. In some implementations, an organ restraint band in accordance with embodiments of the present disclosure comprises wire reinforcement, such as along edge portions of the band. Such reinforcement may help avoid concentrated tension of the band along a central portion of the band. Reinforcement wire may comprise for example, shaped Nitinol support strips and/or edge bands (not shown). Furthermore, in some implementations, restraint bands in accordance with the present disclosure comprise a tightening mechanism for adjusting the tension and/or pressure of the band on the atria or other organ. For example, in certain embodiments, an organ restraint band includes a plurality of lace holes (e.g., 450, 451), such as along a central axis of the restraint band, wherein a lace, ribbon, cord, tie, or other type of line may be used to draw the portions of the band together in a wrapped configuration around the heart or other organ. Tension adjustment mechanisms and methods are disclosed in greater detail below. As described, the band 430B may comprise fluid channels therein for providing pressure/structure adjustment for the band and/or cooling fluid circulation for inflammation reduction or prevention.

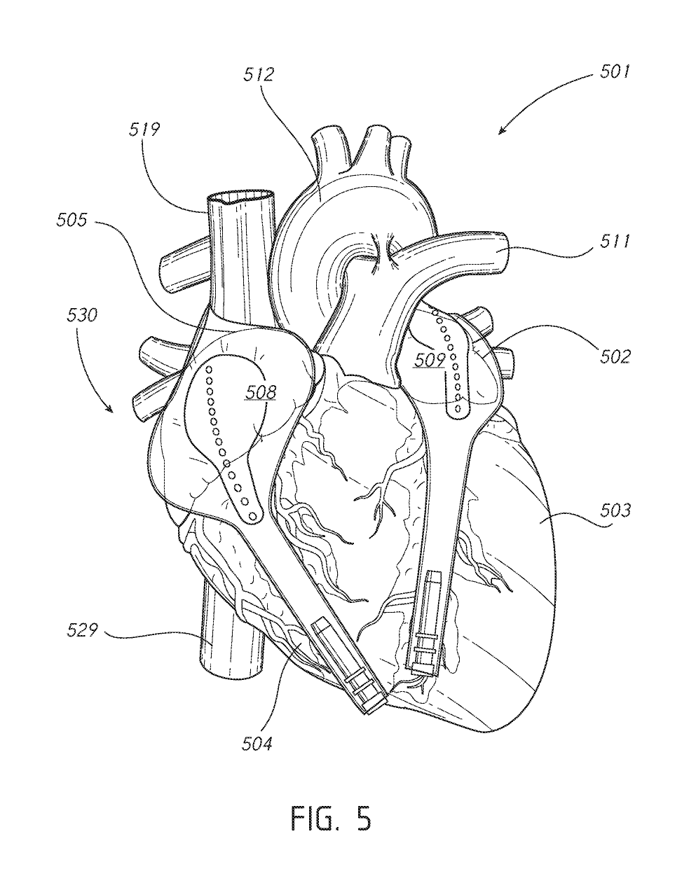

FIG. 5 illustrates a perspective view of a heart 501 having an organ restraint band 530, which may be similar in certain respects to the organ restraint device 430B shown in FIG. 3B and described above, wrapped at least partially around the atria (502, 505) of the heart in accordance with one or more embodiments disclosed herein. As shown, the transversely-projecting/extending protrusions of the band 530 may advantageously align with the atria, to thereby provide saucer-like contact pad forms (508, 509) for covering the surface area of the atria, respectively.

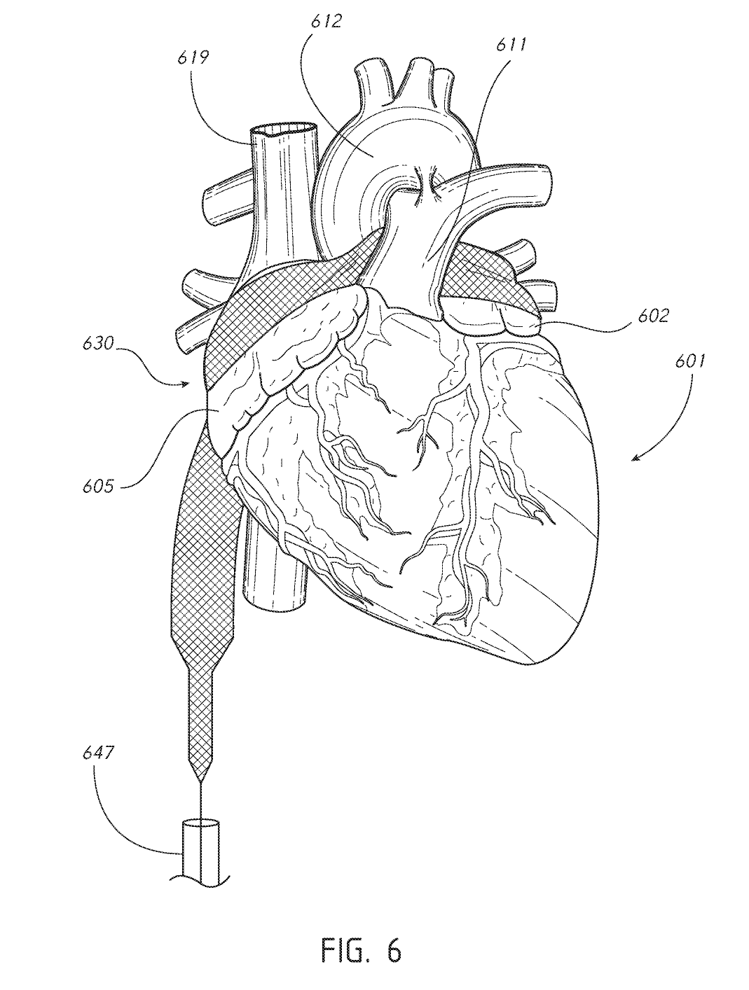

Although some embodiments disclosed herein provide for fluid-filled polymer restraint bands, as described above in connection with FIGS. 3-6, in some embodiments, an organ restraint band, such as a restraint band design for at least partially restraining the atria of the heart by providing external force or pressure thereto, may comprise a mesh or wire band, as shown in the illustrated embodiment of FIG. 6. FIG. 6 illustrates a restraint band 630 comprising a wire or thread mesh or woven configuration. For example, the band 630 may comprise Nitinol memory metal alloy, which may be shaped as desired to fit and/or cover the desired area to be restrained. For example, the band 630 may be wrapped around at least a portion of a heart 601, such that the band 630, or portions thereof, lie in physical contact with the left and/or right atria 602, 605, to provide pressure and restraint thereto.

The memory metal weave 630 may be configured to spring open in some configurations, yet allow for reduction of size for removal of the band through, for example, a catheter 647 or other tool. The memory metal wire to may be configured to fit in the catheter 647, and may be woven with a particular pattern and contoured such that exposure thereto of thermal energy may cause the tube to expand to a desirable size for restraining the atria of the heart 601. In certain embodiments, the expanded size of the woven memory metal (e.g., Nitinol) band may be similar in some respects to the contoured shape of the band 430B illustrated in FIG. 4B and described above. In its expanded state, the memory metal woven band may advantageously have desirable stiffness to prevent stretching of the atria. In certain embodiments, the body temperature of the patient in the region where the band 630 is deployed may be sufficient to cause the band 630 to expand to its desired shape and size. In some embodiments, the woven band 630 may be relatively easily pulled back into the catheter tube 647 for removal thereof. Although FIG. 6 illustrates a wire woven or mesh band 630, should be understood that the principles disclosed herein may be applicable to a band that may be similar to the band 630 of FIG. 6, but rather comprise polymer film, or the like. The band 630 is wrapped around a top portion of the heart, as shown. In certain embodiments, the band 630 is disposed between the aorta 612 and the pulmonary artery 611.

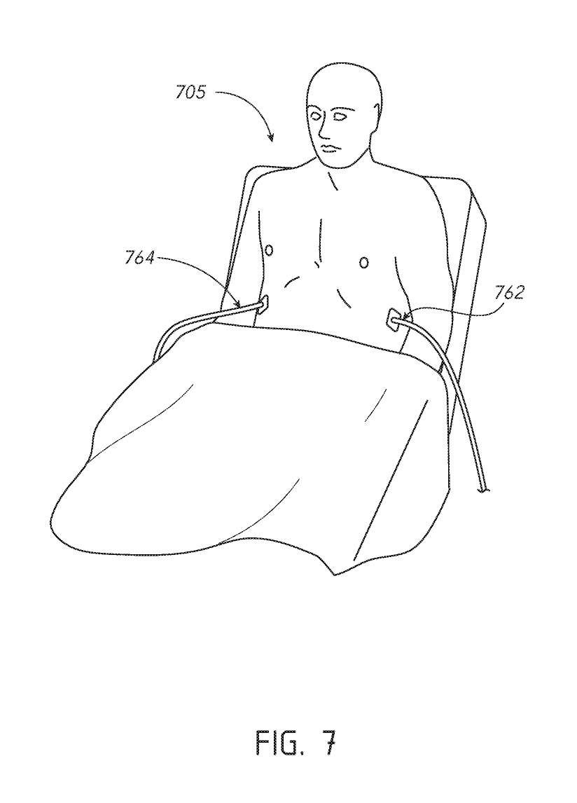

FIG. 7 illustrates a patient 505 in a post-operative state, such as after completion of a vascular surgical operation, such as a cardiac surgery, or the like. In some implementations, it may be desirable for atria restraint bands in accordance with the present disclosure to be retrievable from within a patient a period of time after a surgical operation. That is, it may be desirable to provide access to the implant restraint band after the patient's chest cavity has been closed after surgery. Therefore, in some implementations, devices and methods disclosed herein may utilized chest drainage tubes, such as the chest drainage tubes 764, 762 shown in FIG. 7. For example, an organ restraint band accordance with the present disclosure may be implanted in the patient during surgery, wherein one or more portions thereof may be accessible through one or more chest drainage tubes or other ingress/egress channels/pathways that may remain after completion of the surgical operation, such that the band may be retrieved by pulling the band out of the patient through one or more of the drainage tubes.

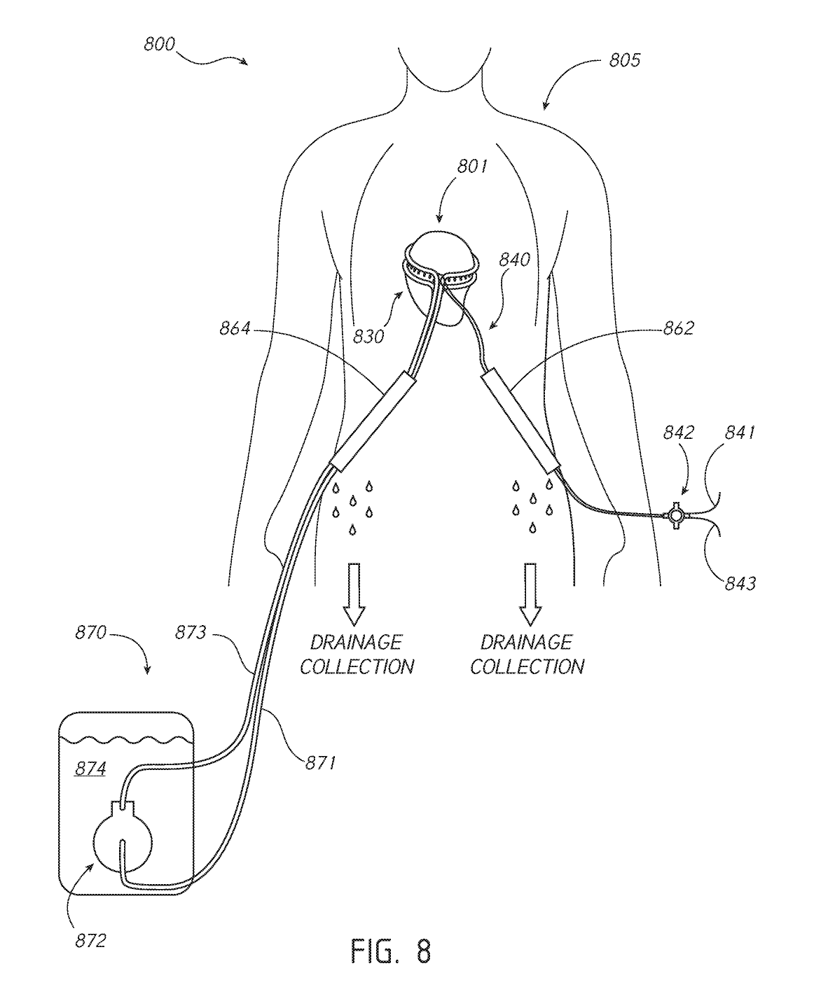

FIG. 8 illustrates a cutaway view of an organ restraint and therapy system 800 in accordance with one or more embodiments. FIG. 8 illustrates an organ 801, such as a heart, of a patient 805 engaged with a restraint band 830 in accordance with one or more embodiments of the present disclosure. The band 830 may be wrapped around the organ 801 and wrapped in such a way as to provide removal access of the band 830 through a first chest drainage tube 864. Although the chest drainage tube 864 is illustrated at a certain position relative to the body of patient 805, it should be understood that such tube may be implanted or disposed on/in any side or area of the patient's body within the scope of the present disclosure. The system further illustrates a tension adjustment system 840, wherein components thereof may have removal access via another chest drainage tube 862. Although removal access is illustrated through separate chest drainage tubes for the restraint band 830 and the tension adjustment system 840, it should be understood that in some implementations, removal access for both elements may be achieved through a single drainage tube.

The therapeutic restraint system 800 shown in FIG. 8 may further include a cooling fluid circuit for propagating a fluid 874 from a fluid reservoir 870 through one or more fluid circuit lines, such as the pump output line 873 coupled to an output of the pump 872, and a pump intake line 871, as shown. The pump 872 may be configured to drive fluid through the pump output line 873, which may be coupled in some manner to the organ restraint band 830, either external to the patient or internal to the patient through the drainage tube 864. The drainage tube 864 may further be utilized for drainage collection of fluid in connection with a surgical operation on the patient 805. The cooling fluid 874 may be circulated through the removable organ restraint band 830 to reduce inflammation of the organ 801, and may be utilized during a period after a surgical operation, such as a five-day initial healing period or process, or other period. In some situations, by circulating cooling fluid through at least a portion of the organ restraint band 830, the organ 801 may thereby be cooled to some extent with respect to at least one or more portions or regions thereof, which may serve to reduce the risk of the patient 805 developing atrial fibrillation. For example, where the patient has undergone a surgery involving incision in one or more of the atria of the heart of the patient 805, such as for a valve repair operation, or the like, the wound area associated with the incision may benefit from the application of a cooling source, such as the organ restraint band 830 when circulating cooling fluid. In some implementations, the cooling fluid reservoir 870 may comprise an ice cooler, or the like, a relatively small pump 872, and/or temperature control circuitry (not shown), which may be utilized to maintain the desired temperature for the fluid circulating in the restraint band 830. In some embodiments, atrial fibrillation and/or fluid overload detection circuitry may also be incorporated.

The therapeutic restraint system 800 shown in FIG. 8 may further include a tension adjustment assembly or system 840, which may be configured to be manipulated by an operator external to the patient to increase or reduce tension of the band 830 as wrapped around the organ 801. The tension management system 840 may include a tension adjustment line locking mechanism 842, which may be lockable and unlockable by an operator externally to the patient. The band 830 is shown wrapped around the organ 801 (e.g., heart) in a generic configuration. However, should be understood with further reference to the remainder of the present disclosure, that the organ restraint band 830 may advantageously be wrapped such that portions thereof provide tension to one or more of the atria of the heart of the patient 805.

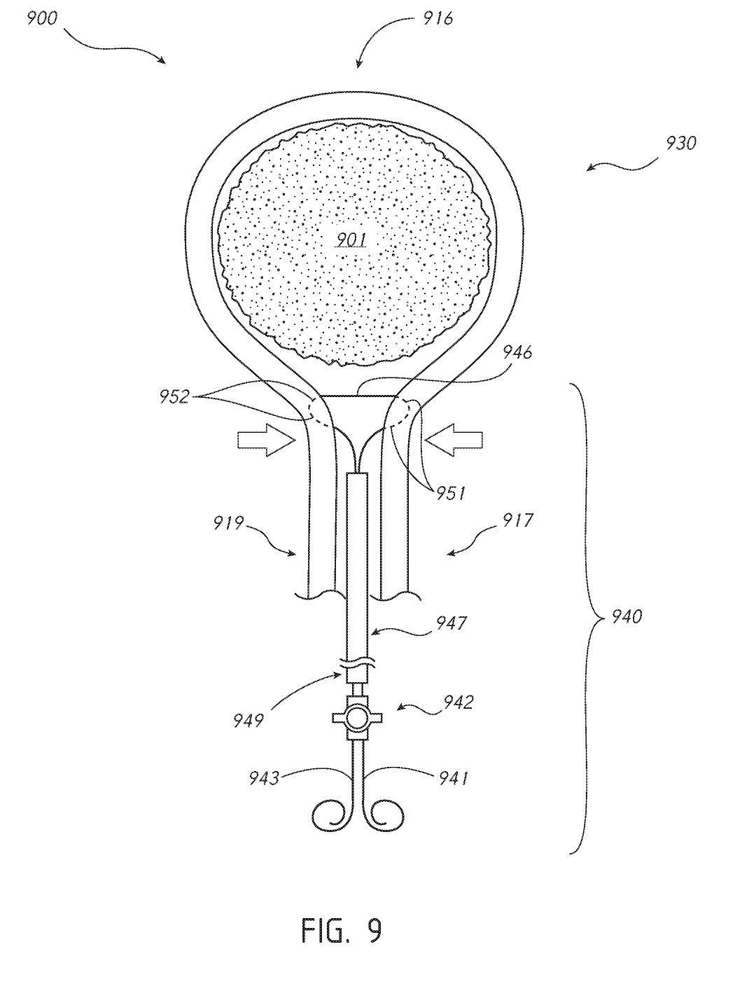

FIG. 9 illustrates a top-down view of an organ restraint and therapy system 900 comprising a removable organ restraint band 930 in accordance with one or more embodiments of the present disclosure. The organ restraint band 930 may be wrapped around an organ 901, such as a heart, as described herein. The diagram of FIG. 9 illustrates a tension adjustment system 940, which can include one or more laces for binding or drawing first 917 and second 918 ends or sides/halves of the organ restraint band 930 to one another to thereby tighten the wrap of the band 930 around the organ 901 to provide increased pressure on one or more areas or portions of the organ 901. For example, with respect to a heart, the band 930 may be tightened to provide increased pressure against one or more of the atria of the heart, to thereby reduce or prevent atrial stretching. Furthermore, with respect to embodiments incorporating cooling fluid circulation in the band 930, contact and/or proximity of the band 930 to the atria may reduce inflammation associated therewith, thereby further reducing the risk of atrial fibrillation. In some implementations, introduction or evacuation of fluid to or from the band 930 may serve to adjust pressure and/or tension in the band 930. Although certain embodiments are described as including a lace, it should be understood that such embodiments may utilize any type of line or cord.

In some embodiments, the restraint band 930 may have one or more lace holes/apertures therein (not shown). A lace 946 may be fed through a compression tube or catheter 947 from a position external to the patient, wherein the thread may be run/passed through the compression tube 947 and exit at distal end thereof, and further be threaded through one or more lace holes 951 of a first side/half 917 of the band 930. The lace or thread 946 may further be threaded through one or more lace holes 952 of a second side/half 919 of the restraint band 930 that has been wrapped around the organ 901, wherein the lace may further be fed back through the compression tube 947 and ultimately exit an external distal end 949 of the compression tube 947, such that the first and second ends/portions 943, 941 of the lace may be accessible external to the patient.

In some embodiments, the portions of the lace 943, 941 present external to the patient, as illustrated, may be incorporated with or threaded through a lace locking mechanism 942, which may be configured to cinch or otherwise lock the laces portions 943, 941, in a relative position to one another, which may serve to hold a desired tension in the band around the organ 901. For example, as one or more of the thread portions 943, 941 are drawn or pulled away from the restraint band, such action may serve to draw the threaded portions of the band 930 together, as shown, which may serve to tighten the band 930 around the organ 901. Therefore, the tension adjustment system 940 may allow for an operator to set a desired pressure or tension of the band 930 around the organ 901. Although certain embodiments are described herein in the context of a lace-threaded or tightened tension adjustment system, it should be understood that any lace, thread, string, cord, ribbon, or other type of line may be used to draw the opposing sides of the restraint band 930 together to thereby increase the tension or pressure thereof around the organ 901. In some embodiments, the lace holes/apertures (not shown) may be incorporated in non-fluid filled portions or sections of the band 930. In some embodiments, the lace locking mechanism 942 may comprise a clasp or slide structure having an adjustable locking feature, which may be engaged to secure the lace portions running therethrough. For example, the adjustable locking component of the lace locking mechanism 942 may be rotatably adjustable, which may engage the locking and unlocking functionality of the locking mechanism. In certain embodiments, the locking mechanism 942 comprises a hemostat. Adjustment of the lace tension system may serve to tighten or loosen the restraint band 930, as needed. During removal of the locking system, in some embodiments, the locking mechanism can be unlocked and the entire lace can be removed from within the patient by pulling on either end of the external lace ends 943, 941.

In some implementations, an atrial restraint band in accordance with one or more embodiments of the present disclosure may be wrapped around the heart, wherein at least a portion of the band is disposed or routed within/through a natural anatomical pocket, or passageway, between the intertwined aorta and pulmonary artery on one side, and the atria on the other side. For example, generally there may be no connective tissue between these structures, thereby providing a groove passageway or pocket through which an organ restraint band in accordance with the present disclosure may be passed on the posterior side of the heart to allow for the band to be wrapped around and/or cover at least part of one or more of the atria of the heart. By securing the organ restraint band in the natural anatomical pocket described herein, the need for suturing or other fixing our attachment of the restraint band to the heart or surrounding tissue may be eliminated or at least partially obviated. The term “pocket” is used herein according to its broad and ordinary meaning, and may refer to any type of passageway, groove, cavity, corridor, path, pathway, or the like, through or over which an organ restraint band in accordance with embodiments of the present disclosure may be drawn, routed, or passed.

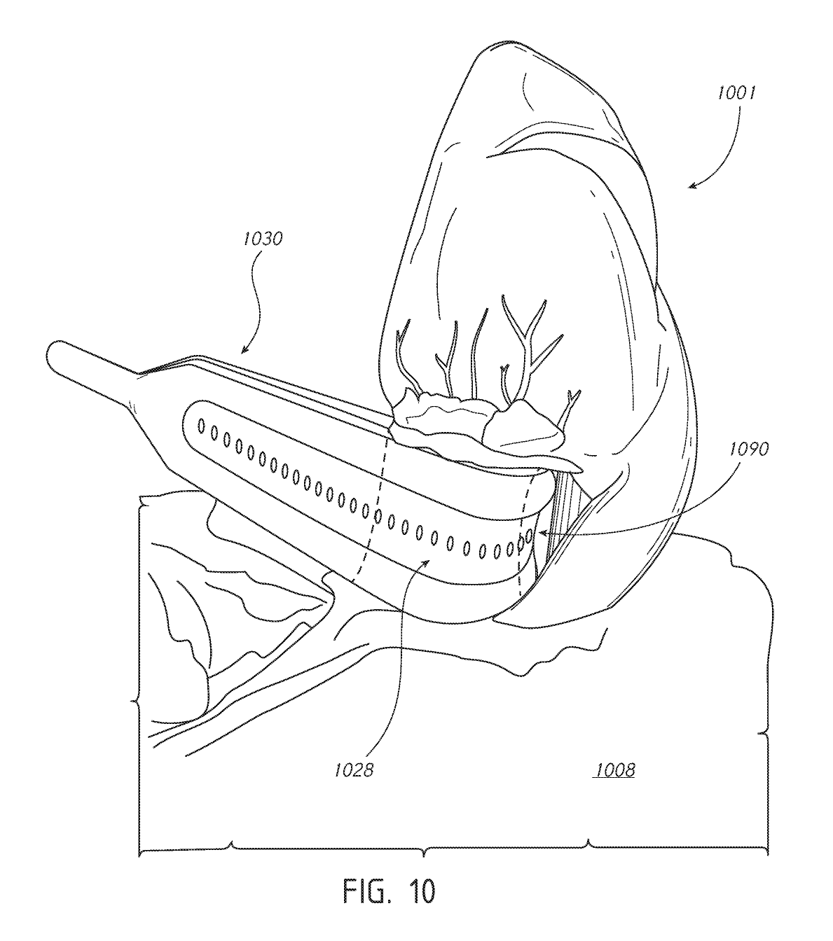

FIG. 10 illustrates a side view of an inverted heart 1001, showing the described pocket or passageway 1090 through which an organ restraint band may be routed. With respect to the view and/orientation of FIG. 8, the pocket 1090 may have an upper natural restraint of one or both of the atria of the heart, and a lower natural restraint of the aorta, trachea, inferior vena cava, and/or other connective tissues 1028, such as tissues associated with the lungs 1008. By utilizing the pocket described herein, atrial restraint may be accomplished using a simple restraint band 1030 in some implementations. The surgeon may carefully draw the restraint band through the pocket 1090 in such a manner as to avoid substantial injury or damage to the heart tissue and components. The pocket/groove 1090 can help to stabilize the position of the band 1030 around the heart 1001, such that movement thereof is at least partly restricted. That is, the pocket/groove 1090 may serve to anchor the band 1030 in a desired position and prevent the band 1030 from undesirably migrating after implantation.

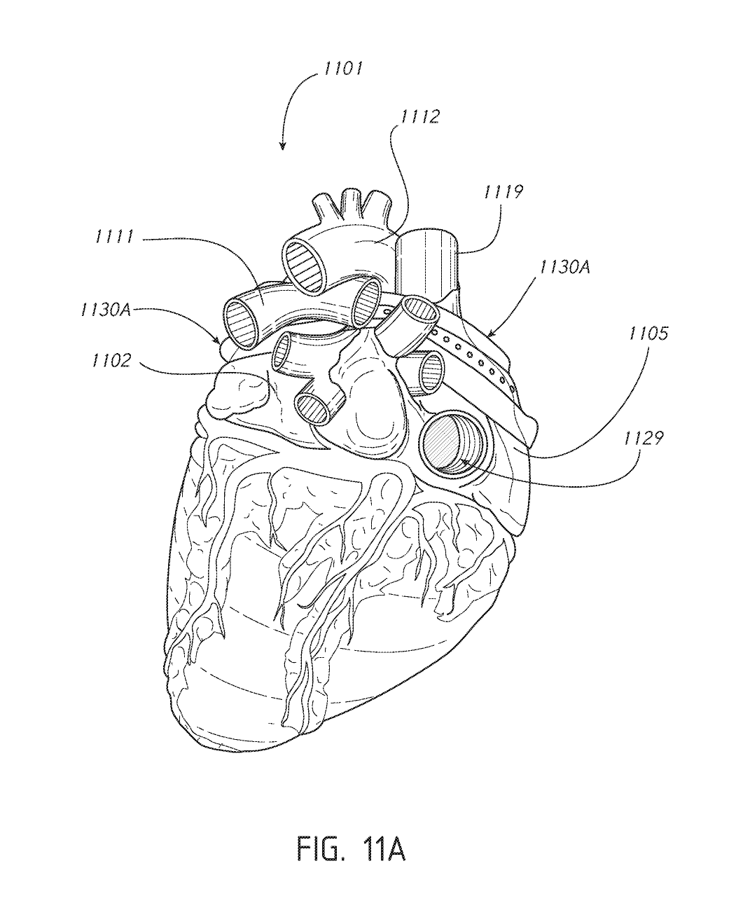

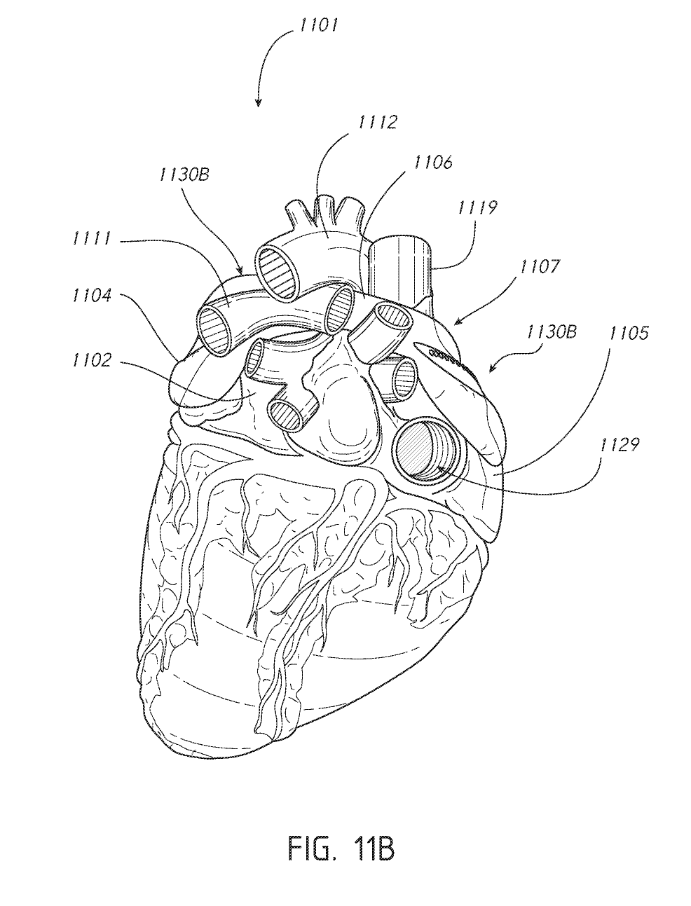

FIG. 11A illustrates a posterior surface view of a heart 1101 having an atria restraint band 1130A routed through the natural anatomical pocket shown in FIG. 10 and described above. The portion of the restraint band 1130A shown corresponds to a medial longitudinal tissue contact portion of the restraint band. The restraint band 1130 may further comprise distal end portions (not shown) as described herein, which may be wrapped around to the anterior of the heart 1101. With respect to the view of FIG. 11A, the band 1130A can be routed in the illustrated pocket behind the pulmonary artery 1111 and the aorta 1112, and in front of, or on, the atria 1102, 1105.

FIG. 11B illustrates another embodiment of an atria restraint band 1130B routed through the natural anatomical pocket between the aorta and/or pulmonary artery and the atria. With reference to FIG. 11B, as described above in connection with FIG. 4B, a restraint band 1130B may comprise a first expanded atria restraint portion 1104 configured and implemented to restrain the left atrium 1102, and a second expanded atria restraint portion 1107 configured and implemented to restrain the right atrium 1105. The restraint band 1130B may further comprise a central recessed notch/groove portion 1106, which may have a reduced width relative to the expanded portions 1104, 1107 to accommodate the atria 1112, superior vena cava 1119, and/or other anatomy of the heart, such that such anatomical components/members may advantageously not be occluded, deformed or displaced to an undesirable degree. That is, the shape of the band 1130B may have a cut-out form to avoid undesirably deforming or displacing blood vessels coupled to the heart that are disposed proximate to the wrapped restraint band 1130B. In certain embodiments, the groove 1106 allows for the aorta 1112, superior vena cava 1119, and/or pulmonary artery 1111 to pass or fit between the transverse projections associated with the expanded atria contact portions 1104, 1107 of the band 1130B.

### Organ/Atria Restraint Band Implantation

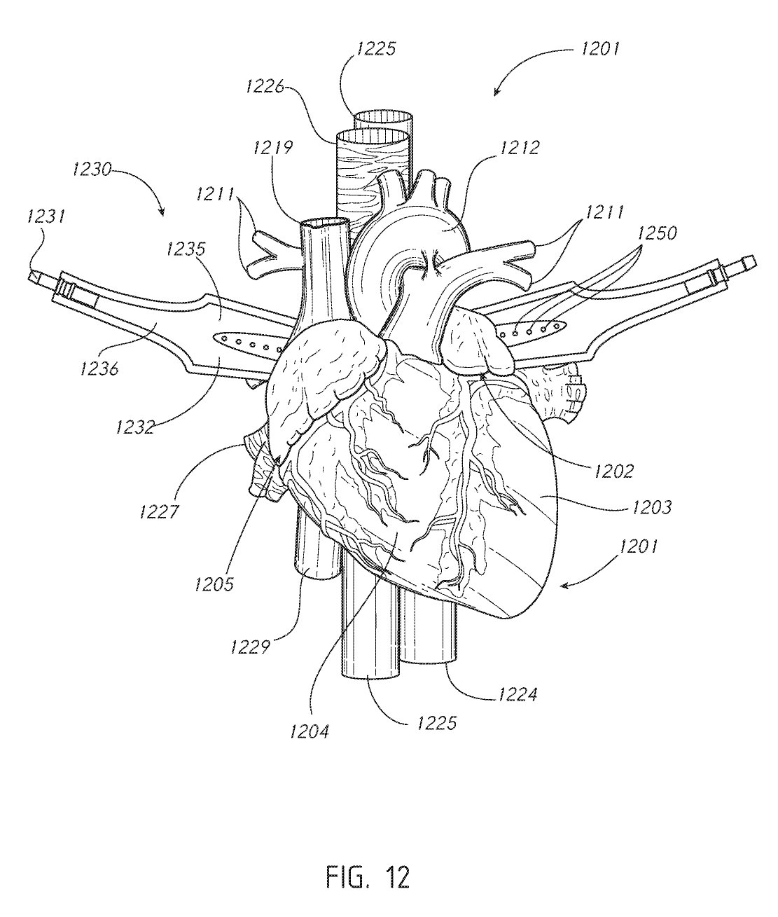

FIG. 12 illustrates an anterior view of a heart 1201 having an atria restraint band 1230 passed behind the heart 1201 to allow for wrapping of the restraint band 1230 over the atria (1205, 1202) of the heart 1201 in accordance with one or more embodiments disclosed herein. For example, the restraint band 1230 may be routed through the anatomical pocket shown in FIGS. 10, 11A, and 11B, and described above. Methods and processes for implanting an atria restraint band in accordance with embodiments disclosed herein may involve, by a surgeon or operator, passing a first end (e.g., 1236) of the restraint band 1230 behind the heart 1201 and through the pocket described above, or through/over any other suitable or desirable passageway or path behind the heart. In some embodiments, once the band 1230 has been arranged behind the heart as shown, one or more sutures, adhesives, or other fasteners or tools may be used to fix at least a portion of the band 1230 to the heart tissue or surrounding tissue. With respect to the particular view of FIG. 12, the process of passing the restraint band 1230 behind the heart may be performed from left to right or from right to left, and either end of the band may lead the band 1230 when passing the same behind the heart 1201. In some implementations, the restraint band 1230 may be implanted in the patient in connection with a surgical operation in which the heart, and/or surrounding anatomy, of the patient is operated on.

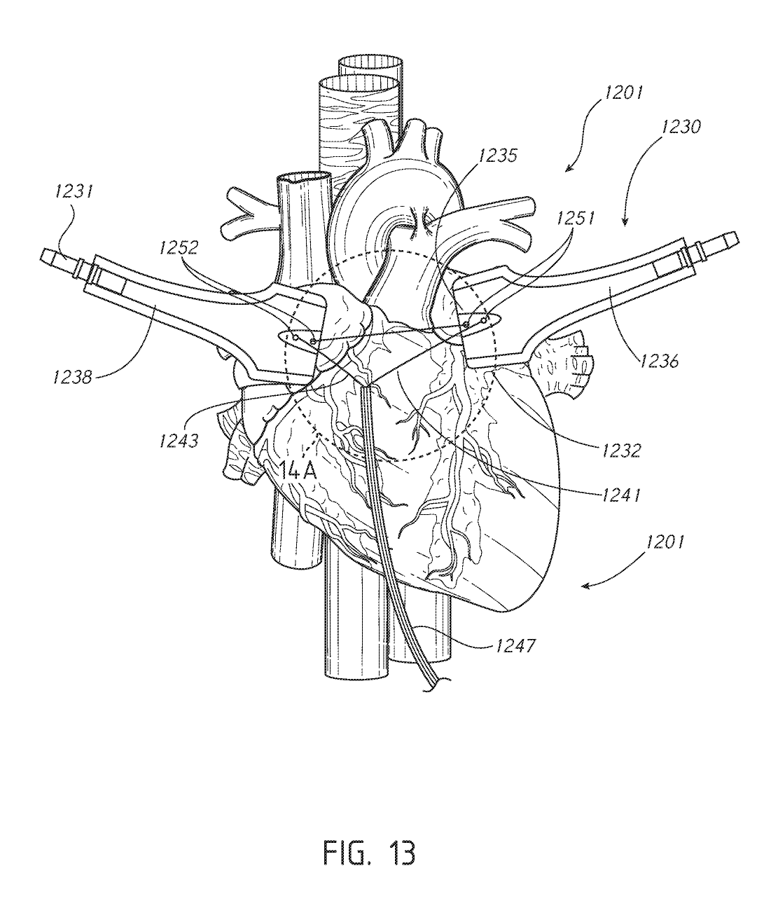

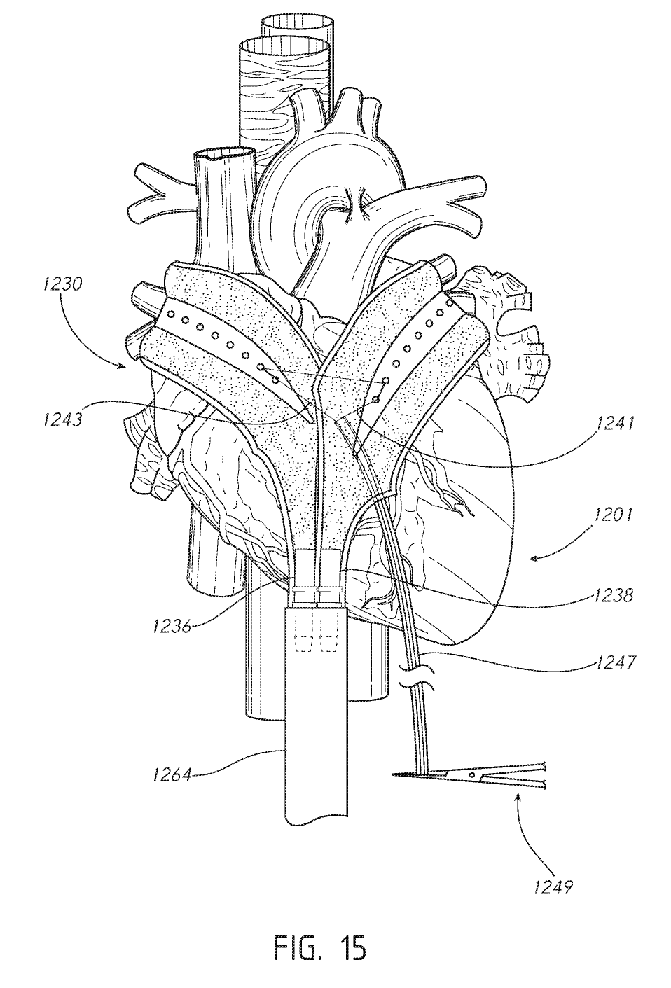

FIG. 13 provides a view of the heart 1201 illustrated in FIG. 12 and described above at a subsequent stage or step of the process of implanting the restraint band 1230 over/around the atria of the heart 1201. In accordance with some embodiments, the restraint band 1230 may comprise one or more holes/apertures for threading of lace, or other type of line, therethrough. For example, with respect to a wrapped-around configuration of the restraint band 1230 as shown in FIG. 15, the first side or half of the restraint band 1230 may comprise a first plurality of lace holes 1252, while the other side or half of the restraint band 1230 may comprise a second plurality of lace holes 1251. The process of implanting the restraint band 1230 may involve introducing a compression tube 1247 into proximity with the heart 1201 and/or restraint band 1230, wherein a lace, cord, ribbon, or type of line may be fed through and/or withdrawn from a local, or internal, distal end portion of the compression tube. The process may further involve treading the lace or other type of line through one or more of the lace holes of one side of the restraint band 1230 (e.g., the lace holes 1251), and further across to the other side of the restraint band and through one or more lace holes associated therewith (e.g., lace holes 1252). The lace 1243 may then be reintroduced into the compression tube 1247 and drawn therethrough such that the end thereof may be withdrawn from external distal end (not shown) of the compression tube.

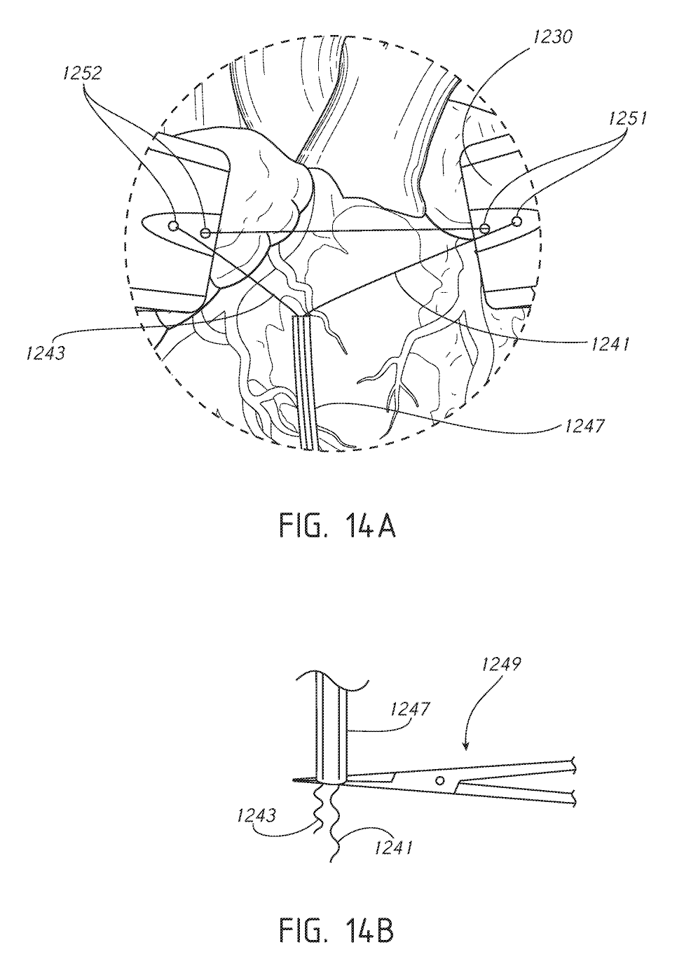

FIG. 14A illustrates a close-up view of the portion of the diagram of FIG. 13 showing the lacing/threading of the restraint band 1230 to provide tension thereto. Furthermore, FIG. 14B illustrates a close-up view of a remote, or external, distal end portion of the compression tube 1247 shown in FIG. 13, wherein such distal end portion may be disposed outside of the chest cavity and/or body of the patient being operated on. In some implementations, as shown in FIG. 14B, the lace portions 1243, 1241 protruding from the remote/external distal end of the compression tube 1247 after the restraint band has been laced together may be fixed or locked in some manner, such as through the use of a locking mechanism of some kind 1249. For example, as illustrated, a hemostat device may be used to hold the lace portions in a relative locked position. Alternatively, any type of clasp or other locking device/mechanism, such as a stopcock-type clasp, may be used within the scope of the present disclosure. That is, any tool, device, or mechanism may be implemented that allows for fixing of relative physical positions of portions of the lace used to tie the two sides/halves of the restraint band together. Prior to locking the tension of the lace, the desired tension for the restraint band 1230 may be achieved by applying tension to, or drawing/pulling, one or both portions 943, 941 of the lace. Once the desired tension is reached, the locking device 1249 may be used to lock the lace at the desired tension.

FIG. 15 illustrates a subsequent stage of the process for implanting and securing the restraint band 1230, wherein end portions 1236, 1238 of the restraint band may be fed into a tube or conduit 1264, which may be associated with or integrated with a chest drainage tube, as described above. In some implementations, the depiction of FIG. 15 illustrates a substantially completed implantation process of the restraint band 1230, wherein the restraint band 1230 is placed at least partially over the left and right atria, with distal tails thereof nested within chest drainage tube 1264 (full length of fluid tubing not shown), with the restraint band 1230 drawn relatively tightly around both atria. Furthermore, the band tensioning system is illustrated in a locked position, providing fixed, consistent tension for the band 1230 to provide desirable restraint of the atria. The process for implanting the restraint and 1230 may involve injecting fluid into one or more ends of the restraint band 1230. For example, a first end of the restraint band (e.g., 1238), may receive cooling fluid from a fluid source, wherein such fluid may enter the fluid channels of the restraint band (e.g., via a fluid inlet channel of the restraint band 1230) and circulate along a length thereof, and ultimately exiting an opposite end (e.g. 1236) of the restraint band 1230 (e.g., through a fluid outlet channel of the restraint band 1230). Although certain embodiments are disclosed herein wherein fluid circulates through the restraint band and a fluid circuit, in some implementations, fluid may be introduced into the restraint band and maintained without circulation thereof. Furthermore, the volume of fluid injected into the restraint band may be selected and managed in order to provide a desirable amount of cooling and/or pressure or structure for the restraint band 1230.

As described above, the atria restraint band 1230 may be maintained in the wrapped configuration around the atria for a period of time following a vascular operation, such as a period of 3-5 days or longer. After the desired period of time has elapsed, the restraint band 1230 may be withdrawn from the patient's chest cavity. Such withdrawal of the restraint band may advantageously be performed without the tissue of the patient proximal to the restraint band suffering injury or other damage according to restraint band removal processes disclosed herein.

### Organ/Atria Restraint Band Removal

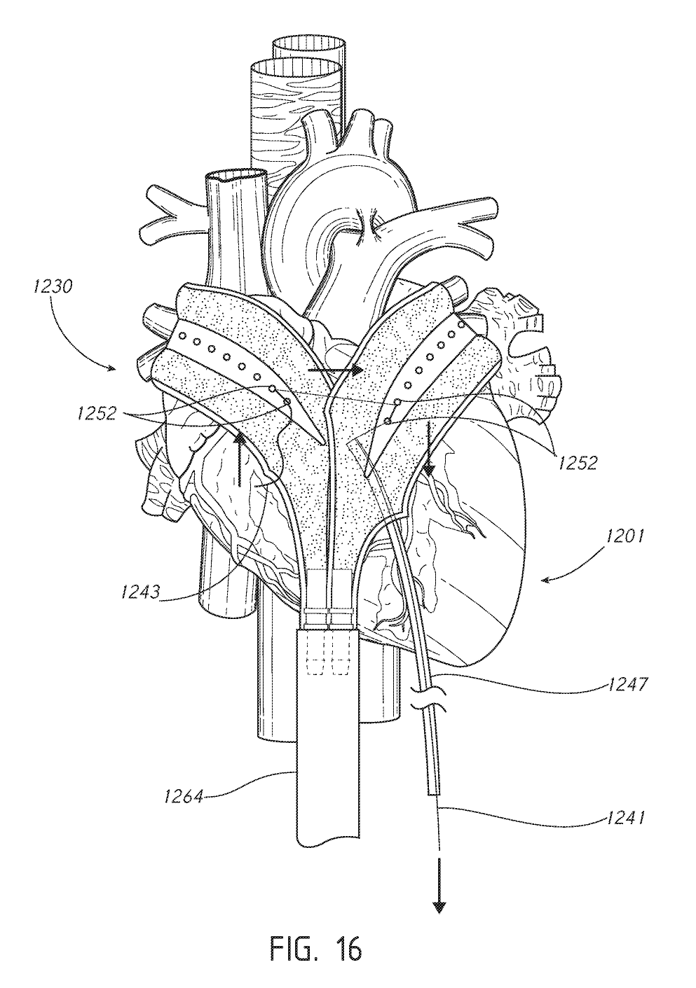

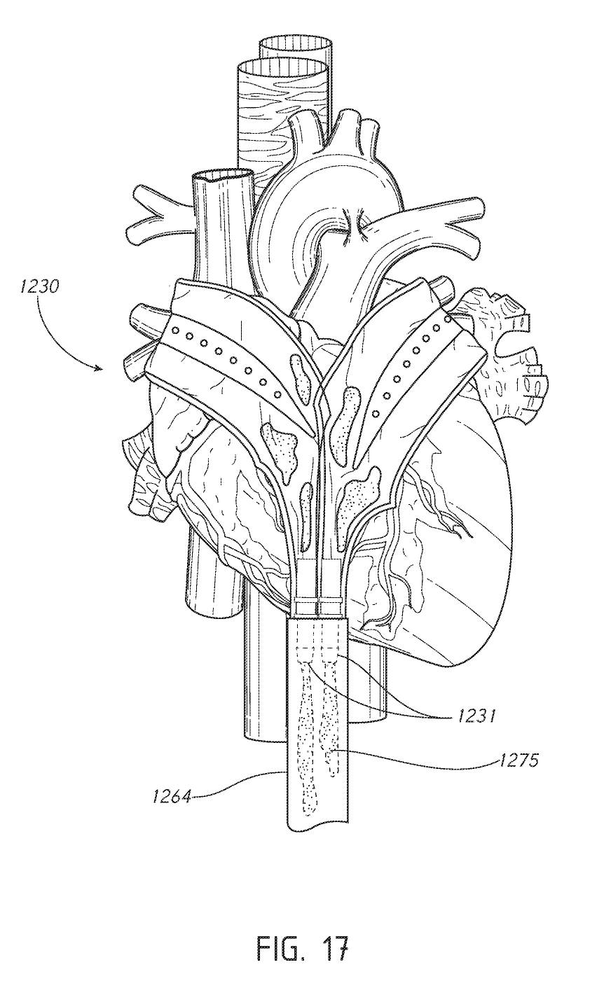

FIGS. 16-18 illustrate stages of a restraint band removal process according to one or more embodiments disclosed herein. The diagrams of FIGS. 16-18 correspond to the heart 1201 and restraint band 1230 shown in FIGS. 12-15. In some implementations, the process for removal of the restraint band 1230 involves, first, disassembling the tension system holding the restraint band in its fixed position. For example, this may be accomplished by unlocking the lace/line-locking mechanism (e.g., hemostat, clasp, or other lace/line-locking component), and withdrawing the lace (or other type of line) and the lace/line compression tube 1247 from the chest cavity of the patient. For example, as shown in FIG. 16, the process may involve pulling on one end of the lace to de-thread the lace from the restraint band 1230, and fully withdrawing the lace from the implantation site. The lace may be withdrawn after or prior to removal of the lace compression tube 1247.

With reference to FIG. 17, the process of removing the restraint band 1230 may further involve evacuating fluid 1275 (e.g., cooling fluid) from the restraint band 1230 that may have been maintained or circulated therein. For example, the fluid 1275 may be withdrawn from one or both ends 1231 of the restraint band 1230, and through the drainage tube 1264.

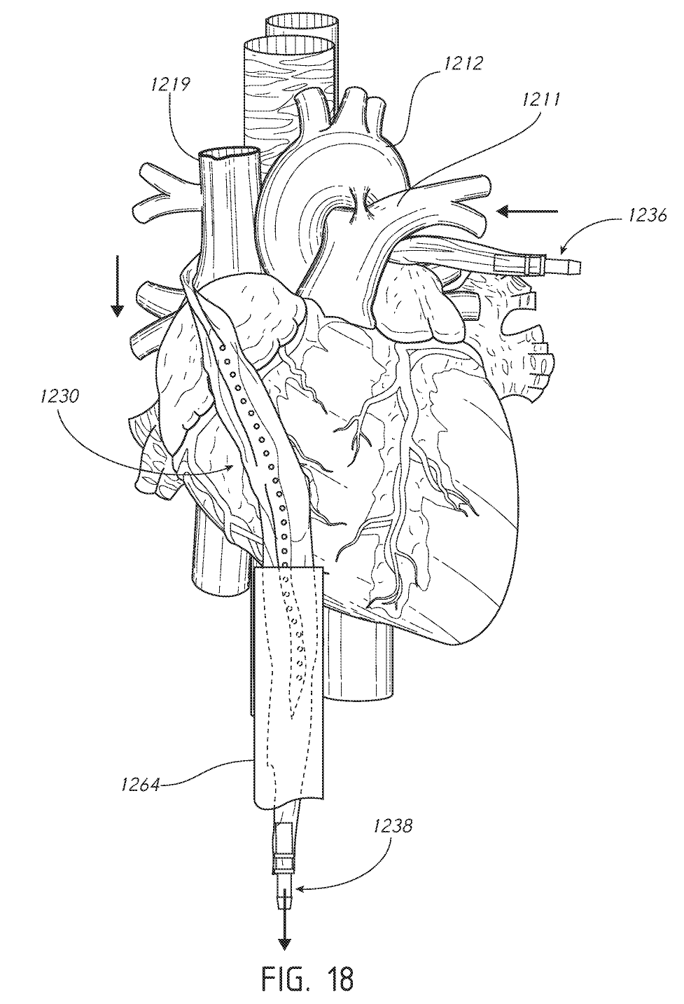

With reference to FIG. 18, the process for removing the restraint band 1230 may further involve pulling or withdrawing one end (e.g. end 1238) of the restraint band 1230, such that the other end (e.g. 1236) may likewise be pulled up and around the heart and back down through the chest drainage tube 1264. For example, the restraint band 1230 may be pulled through the anatomical pocket behind the heart, as described above. The end of the restraint band 1230 may be pulled until the restraint band 1230 is fully withdrawn through the chest tube 1264 and out of the patient therethrough. In some embodiments, the process for removing the restraint band 1230 may further involve removing the chest drainage tube 1264, which may no longer be needed for further treatment of the patient.

With respect to the lace/line 1241 and/or restraint band 1230, one or more ends or portions thereof may be cut externally to the chest cavity in order to prevent such ends/portions, which may be at least partially unsterile, from being pulled through the chest cavity and/or around the heart. For example, ends of the line 1241 and/or band 1230 that protrude from the chest access tube(s) may be cut or broken off prior to removal of such components in accordance with the processes described above. Additionally, or alternatively, ends or portions of the lace/line 1241 and/or restraint band 1230 may be treated with antiseptic to manage or prevent infection which may result from contact of unsterile portions thereof with internal tissue of the patient.

### Additional Organ/Atria Restraint Embodiments

In some implementations, atrial restraint may be achieved through the use of synthetic and/or memory metal (e.g. Nitinol) mesh. For example, restraint mesh may comprise a restraint patch, such as a Silastic patch. Such patch may be trimmed or customized to fit a particular patient's atria or atrium. In some embodiments, an atrial restraint patch may be sutured in place over the atrium. Atrial restraint patches may advantageously comprise bio-resorbable material, such that patch need not be removed from the patient after its useful life. In some embodiments, restraint is achieved through the use of polymer film, which may be deposited or applied to the regions of the atria that are desired to be restrained. However, such films may not provide desirably uniform restraint force in some implementations.

Use of synthetic mesh may advantageously provide desirable restraint, and may be formed to fit a desired shape atria. Mesh restraint patches or bands may be trimmed or cut using scissors or other tools, such that a surgeon may be able to fit or trim the patch/band him or herself at the time of operation. A mesh restraint band may utilize similar cinching and/or tensioning mechanisms to those described above with respect to atrial restraint bands. In some embodiments, a mesh band may comprise a plastic mesh material, which may provide improved flexibility. However, coarse mesh restraint bands may present relatively rough edges, which may undesirably snag or otherwise further damaged tissue proximate thereto, particularly during implantation and/or removal thereof.

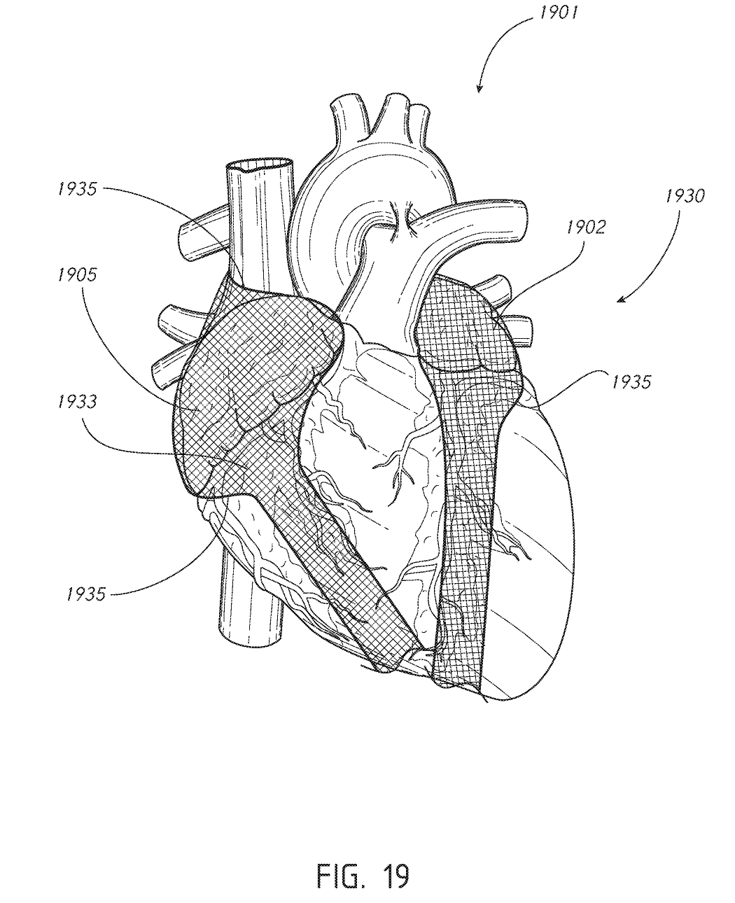

In some implementations, synthetic mesh may be enhanced with shaped edge banding structure, such as memory metal (Nitinol). FIG. 19 illustrates a perspective view of a restraint band 1930 draped over the atria 1902, 1905 of a heart 1901 in accordance with one or more embodiments. The restraint band 1930 comprises a mesh portion 1933, as well as edge banding portions 1935. The rigidity and shape of the edge banding 1935 may advantageously assist in draping/covering a larger portion of the target atrium, and providing more even pressure over the coverage area of the restraint band 1930.

### Combined Ventricle and Atria Restraint Device

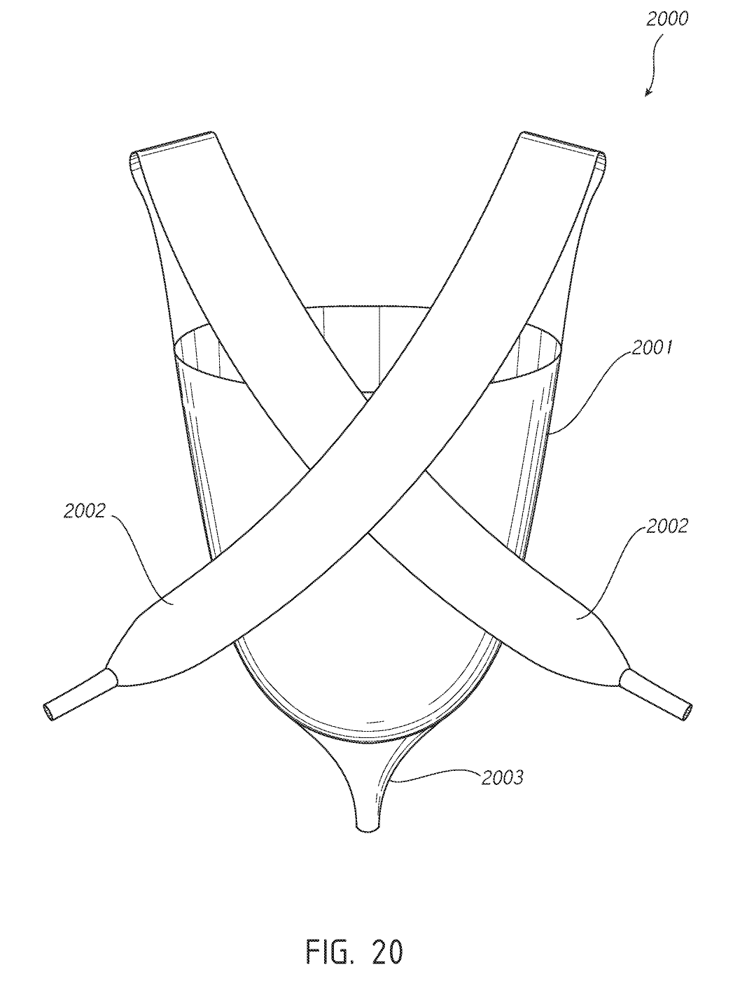

In some medical treatments, the ventricles of the heart may be restrained to prevent or treat mitral valve dysfunction. For example, by restraining the expansion of the ventricles, mitral valve competency may be facilitated or promoted, which may in turn improves the perfusion of the heart, which may depend at least in part on the ability of the ventricle to squeeze adequately to produce the desired output. In some implementations, the present disclosure provides devices and methods for providing combined ventricular and atrial restraint for a heart of the patient. Such devices may advantageously provide prevention for both atrial stretch and/or fibrillation, as well as ventricular dilation. FIG. 20 illustrates a restraint device 2000 that comprises a pouch portion 2001 that is configured to surround and/or constrain the left and/or right ventricles of a heart. The pouch portion 2001 may serve to constrain the left and/or right ventricle of the heart to prevent the same from dilating undesirably, which may help improve cardiac output, among other possible benefits.

The device 2000 further comprises one or more straps 2002 that may be coupled to or integrated with the pouch component 2001. The strap components 2002 may be configured to fold over the right and left atrium of the heart to provide atrial restraint as described in detail herein. In certain embodiments, distal ends of the straps may be connected to and/or fed through chest tube(s) implanted in a patient, which may allow for external manipulation of the straps, thereby providing more or less restraint/pressure to be placed on the atria post-operatively. For example, the ends of the straps may be coupled to a length of cord or other material or means for pulling the straps to thereby tighten them, or to introduce slack into the straps to loosen the pressure provided thereby. In some embodiments, the straps 2002 are configured to pass fully through chest tubes of the patient to allow for fixing and/or manipulation thereof externally when the patient is in a post-operative state.

In some embodiments, the device 2000 further comprises a retrieval tab 2003, which may be used for retrieving the device 2000 post-operatively from the patient, such as in a similar manner to the retrieval process described above in connection with the organ restraint bands disclosed herein. For example, in some implementations, the two straps 2002 may be disconnected from their respective chest tube(s), thereby allowing for the entire device to be pulled out from a chest tube by pulling on the tab 2003 and/or other portion of the device 2000 and thereby extracting the device through the chest tube. For example, the device 2000 may be withdrawn through a lowest chest tube implanted in the patient.

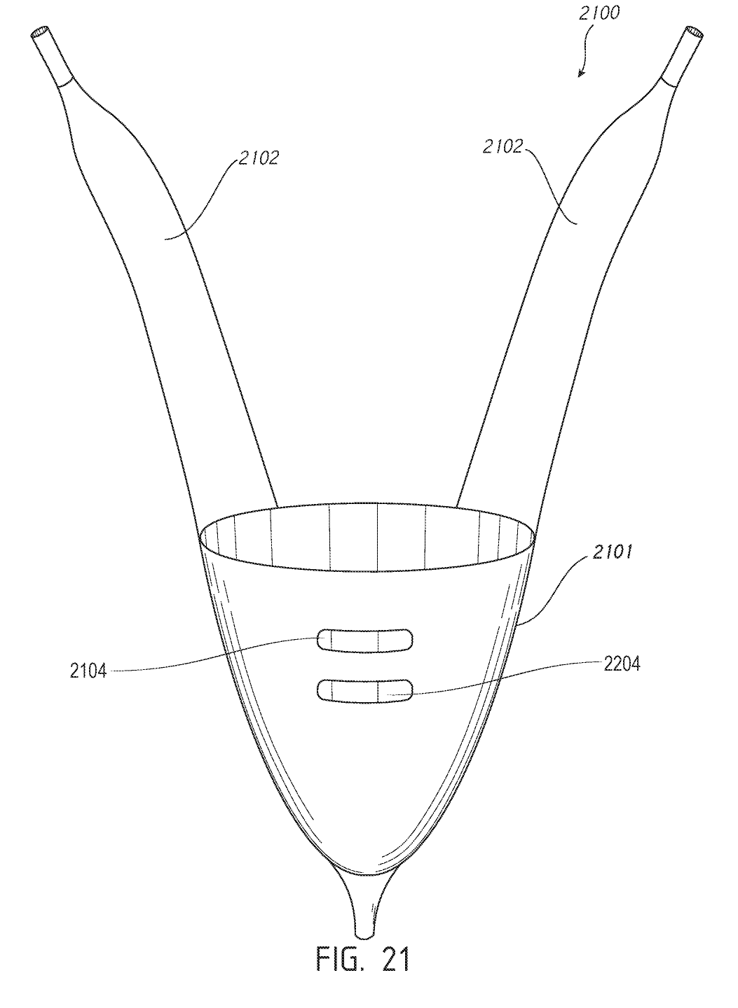

FIG. 21 illustrates a restraint device 2100 that comprises a ventricle restraint pouch 2101 and atrial restraint straps 2102. For example, the restraint device 2100 may represent an embodiment of the restraint device 2000 illustrated in FIG. 20 described above. The device 2100 may further comprise a strap retention feature, which may include one or more slots (e.g., slots 2104, 2204, wherein the straps 2102 may be passed through the slots to secure the straps within the pouch.

In some embodiments, the restraint device 2100 comprises conductive surfaces that may rest at least partially on the tissue of the heart when the device 2100 is implanted about a heart of a patient. For example, conductive surfaces disposed on the strap portions 2102 may be configured to rest on the atria of the heart, or between the pulmonary veins of the heart. Such conductive services may at least partially promote or ensure that the epicardium is at a similar electrical potential, to thereby reduce the risk of generation of recurrent circuits and atrial fibrillation.

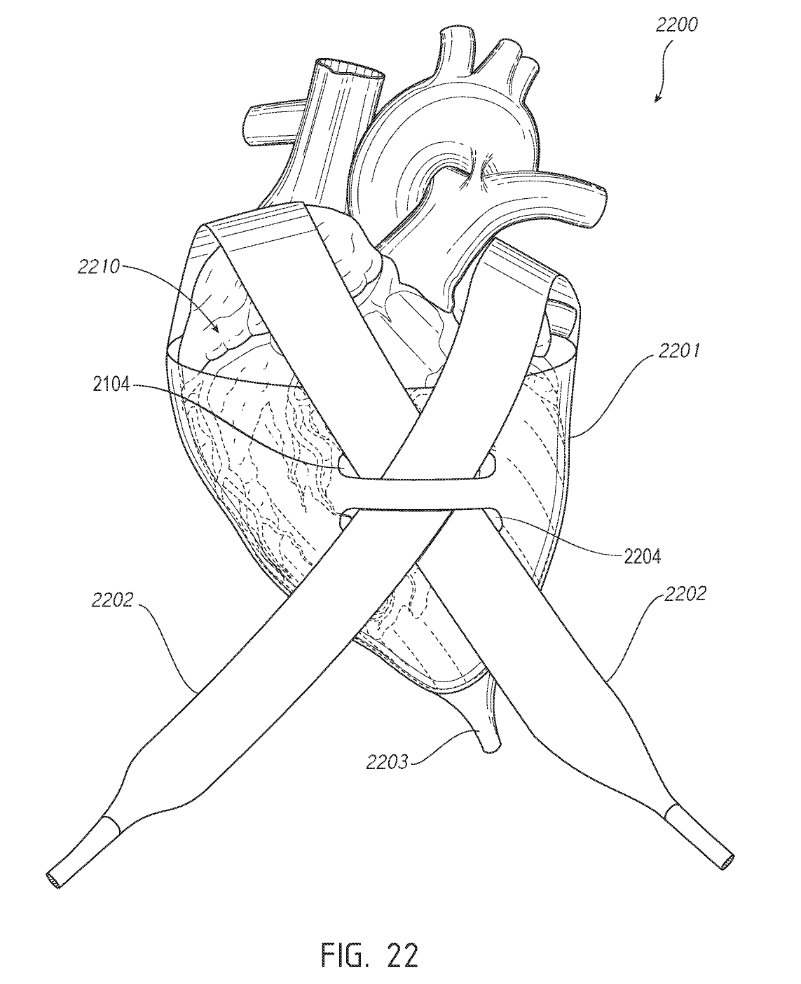

FIG. 22 illustrates a restraint device 2200 as implanted and surrounding a heart 2210 according to one or more embodiments. The restraint device 2200 may represent an embodiment of the restraint device 2000 shown in FIG. 20 and described above. For example, the restraint device 2200 may comprise a ventricle restraint pouch 2201, as well as first and second atrium restraint straps 2202, which may restrain the atria as described above. The straps 2202 are shown secured in a strap retention feature comprising first and second slots 2104, 2204.

In an alternative embodiment, the device 2200 may comprise a band (not shown) that surrounds the atria without the illustrated ventricular pouch 2201. The band may advantageously be adjustable outside of the body when the device 2200 is implanted in the patient.

### Annular Organ/Atria Restraint Device

in some implementations, the present disclosure provides an annular restraint device comprising a drawstring mesh bag having a detachable annular restraint and/or formable plastic tabs for placement around, for example, pulmonary veins, the pulmonary artery, and/or the aorta of a heart. For example, such a device may comprise a simple mesh bag that is configured to be wrapped around the atria. The mesh bag may have associated therewith one or more deformable tabs configured to fit onto or around one or more of the pulmonary veins, aorta, and/or pulmonary artery. In certain embodiments, the device comprises a ripcord component, which may be utilized for enabling retrieval of the device. Furthermore, in some embodiments, the device comprises a cold-injectate lumen. Annular restraint devices in accordance with the present disclosure may provide for relatively easy post-operative retrieval, and/or reduced electrical interference.

In some implementations, annular restraint devices as disclosed herein may provide a drawstring feature configured to cinch the atria, and/or one or more Velcro straps. Furthermore, the annular restraint device may provide a tube or conduit for circulating and/or maintaining cold fluid therein. Furthermore, as referenced above, an annular restraint device in accordance with the present disclosure may provide plastic and/or formable tabs configured to grab or secure to the pulmonary veins and/or aorta. Such tabs may be utilized to avoid certain anatomy and/or navigate the device around various vessels of the heart. Furthermore, such tabs may serve to hold the restraint device in place and help keep the device positioned in a desirable orientation and configuration. In certain embodiments, the mesh bag of the annular restraint device may provide restraint pressure or force on the atria of the heart. The drawstring components may be associated with the clasp or other locking mechanism, which may be local to the device, and not outside of the patient. In some embodiments, the annular restraint device may be configured to allow for the injection of cool gas into the chest cavity proximate to the implanted device, which may help to reduce inflammation and/or provide other benefits.

### Additional Embodiments

Depending on the embodiment, certain acts, events, or functions of any of the processes described herein can be performed in a different sequence, may be added, merged, or left out altogether. Thus, in certain embodiments, not all described acts or events are necessary for the practice of the processes. Moreover, in certain embodiments, acts or events may be performed concurrently.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

What is claimed is:

1. An organ restraint device comprising:
a first end portion including a fluid inlet channel;
a second end portion including a fluid outlet channel; and
a medial body portion coupled between the first end portion and the second end portion, the medial body portion comprising:
one or more fluid channels that couple the fluid inlet channel to the fluid outlet channel; and
a plurality of tension adjustment apertures.

2. The organ restraint device of claim 1, wherein the fluid inlet channel, the fluid outlet channel, and the one or more fluid channels are configured to allow cooling fluid to be introduced into the organ restrain device through the fluid inlet channel, passed through the one or more fluid channels of the medial body portion, and expelled out of the organ restraint device through the fluid outlet channel

3. The organ restraint device of claim 1, wherein the medial body portion comprises an elongate band.

4. The organ restraint device of claim 1, wherein the medial body portion comprises one or more transversely-projecting contours.

5. The organ restraint device of claim 4, wherein the one or more transversely-projecting contours comprise first and second longitudinally-spaced transversely-projecting contours, with a recessed groove therebetween.

6. The organ restraint device of claim 5, wherein the first and second transversely-projecting contours are shaped for contacting left and right atria, respectively, of a heart.

7. The organ restraint device of claim 1, wherein the medial body portion comprises:
first and second longitudinally-aligned transversely-projecting contours that project in opposing directions and form a left atrium contact pad; and
third and fourth longitudinally-aligned transversely-projecting contours that project in opposing directions and form a right atrium contact pad;
wherein the left atrium contact pad is longitudinally offset from the right atrium contact pad.

8. The organ restraint device of claim 1, wherein the medial body portion comprises flexible polymer.

9. The organ restraint device of claim 1, wherein the one or more fluid channels of the medial body portion comprise first and second parallel fluid channels that branch from one or more of the fluid inlet channel and the fluid outlet channel.

10. The organ restraint device of claim 1, wherein one or more of the first end portion and the second end portion comprise a fluid tube connection fitting.

11. A method of treating atria of a heart to prevent atrial fibrillation, the method comprising:
inserting a first end of an organ restraint device into a chest cavity of a patient;
passing the first end of the organ restraint device around a posterior side of a heart of the patient;
wrapping the organ restrain device over one or more of a right atrium and a left atrium of the heart;
threading a tension adjustment line through one or more first apertures associated with a first longitudinal half of the organ restrain device;
threading the tension adjustment line through one or more second apertures associated with a second longitudinal half of the organ restraint device;
drawing the one or more first apertures towards the one or more second apertures by pulling on the tension adjustment line;
inserting cooling fluid into one of the first end and a second end of the organ restraint device; and
expelling the cooling fluid from another of the first end and the second end.

12. The method of claim 11, further comprising applying pressure to the one or more of the right atrium and the left atrium of the heart using the organ restraint device.

13. The method of claim 12, wherein said applying pressure restrains one or more of the left and right atria from expanding beyond 5 mm in diameter.

14. The method of claim 11, further comprising inserting the tension adjustment line into a first end of a compression tube.

15. The method of claim 14, further comprising withdrawing the tension adjustment line from a second end of the compression tube, wherein said pulling on the tension adjustment line involves pulling the tension adjustment line through the compression tube.

16. The method of claim 15, further comprising locking the tension adjustment line externally to the patient.

17. The method of claim 16, wherein said locking the tension adjustment line is performed using a locking mechanism coupled to the tension adjustment line and disposed externally to the patient.

18. The method of claim 11, wherein said passing the first end of the organ restraint device around the posterior side of the heart involves passing the first end through a pocket between an aorta or pulmonary artery of the patient and one or more atria of the heart.

19. The method of claim 11, wherein said drawing the one or more first apertures towards the one or more second apertures at least partially prevents stretching of one or more atria of the heart due to fluid overload associated with a surgical operation.

20. The method of claim 11, wherein said inserting and expelling the cooling fluid reduces inflammation of one or more of the left and right atria to prevent atrial fibrillation.

21. The method of claim 11, further comprising controlling a temperature of the cooling fluid to provide a desired therapeutic effect.

22. The method of claim 11, further comprising nesting the first and second ends of the organ restraint device in one or more chest drainage tubes.

23. A method of removing an organ restraint device from a chest cavity of a patient, the method comprising:
unlocking a tension adjustment line threaded through one or more apertures of an organ restraint device wrapped around one or more of a right atrium and a left atrium of a heart of a patient;
withdrawing the tension adjustment line from a chest cavity of the patient;
evacuating fluid from the restrain device; and
withdrawing the organ restraint device from the chest cavity of the patient by pulling on a first end of the organ restraint device to thereby draw a second end of the organ restraint device around a posterior side of the heart.

24. The method of claim 23, wherein the method is performed while the chest cavity of the patient is closed.

25. The method of claim 23, wherein said withdrawing the tension adjustment line from the chest cavity involves pulling the tension adjustment line through a first chest tube implanted in the patient.

26. The method of claim 25, wherein said withdrawing the organ restraint device from the chest cavity involves pulling the organ restraint device through a second chest tube implanted in the patient.

27. The method of claim 23, wherein the method is performed between three and five days after a surgical operation involving the patient.

28. The method of claim 23, wherein said unlocking the tension adjustment line involves disengaging a locking mechanism external to the patient.

29. The method of claim 28, wherein the locking mechanism comprises a hemostat.

30. An atria restraint band comprising:
a first distal end portion;
a second distal end portion;
a first atrium contact portion positioned along a longitudinal dimension of the atria restraint band, the first atrium contact portion comprising a first protrusion in a first transverse direction, and a second protrusion in a second transverse direction opposite the first transverse direction, the second protrusion being aligned longitudinally with the first protrusion;
a second atrium contact portion positioned along the longitudinal dimension of the atria restraint band, the second atrium contact portion comprising a third protrusion in the first transverse direction, and a fourth protrusion in the second transverse direction, the third protrusion being aligned longitudinally with the fourth protrusion; and
a transversely-recessed groove positioned between the first and second atrium contact portions along the longitudinal dimension of the atria restraint band.

31. The atria restraint band of claim 30, wherein the first and second atrium contact portions are shaped to cover a majority of a surface area of an atrium of a heart.

32. The atria restraint band of claim 30, wherein the transversely-recessed groove is shaped to accommodate the presence of a blood vessel to reduce deformation or displacement thereof when the atria restrain band is implanted in proximity to the blood vessel.

33. The atria restraint band of claim 32, wherein the blood vessel is a superior vena cava.

34. The atria restraint band of claim 32, wherein the blood vessel is a pulmonary artery.

35. The atria restraint band of claim 32, wherein the blood vessel is an aorta.

36. The atria restraint band of claim 30, further comprising an edge support wire associated with an edge portion of the atria restraint band.
